SEQUENCE LISTING (19 sequences)

SEQ ID NO: 1 - protein, 9 aa, Artificial
  synthetic
GGIVTYGKI

SEQ ID NO: 2 - protein, 11 aa, Artificial
  synthetic
GIVTYGKIPYP

SEQ ID NO: 3 - protein, 6 aa, Artificial
  synthetic
GIVTYG

SEQ ID NO: 4 - protein, 8 aa, Artificial
  synthetic
GIVSYGKI

SEQ ID NO: 5 - protein, 8 aa, Artificial
  synthetic
GIVTFGKI

SEQ ID NO: 6 - protein, 8 aa, Artificial
  synthetic
GIVTYGKI

SEQ ID NO: 7 - protein, 7 aa, Artificial
  synthetic
GATYGKI

SEQ ID NO: 8 - protein, 8 aa, Artificial
  synthetic
GIITYGKI

SEQ ID NO: 9 - protein, 7 aa, Artificial
  synthetic
GILTYGK

SEQ ID NO: 10 - protein, 8 aa, Artificial
  synthetic
GIVTYGKI

SEQ ID NO: 11 - protein, 8 aa, Artificial
  synthetic
GLVTYKKI

SEQ ID NO: 12 - protein, 25 aa, Artificial
  synthetic
YEIVTYGKIPYPGRTNADVMTALSQ

SEQ ID NO: 13 - protein, 18 aa, Artificial
  synthetic
ANLMKTLQHDKLVRLYAV

SEQ ID NO: 14 - protein, 9 aa, Artificial
  synthetic
LYAVVTREE

SEQ ID NO: 15 - protein, 19 aa, Artificial
  synthetic
ITEYMAKGSLLDFLKSDEG

SEQ ID NO: 16 - protein, 7 aa, Artificial
  synthetic
SLLDFLK

SEQ ID NO: 17 - protein, 7 aa, Artificial
  synthetic
GIVTYGK

SEQ ID NO: 18 - protein, 7 aa, Artificial
  synthetic
KGYTVIG

SEQ ID NO: 19 - DNA, 30 bp, Artificial
  synthetic
atgggatgtataaaatcaaaagggaaagac

(19) United States
(12) Patent Application Publication
(10) Pub. No.: US 2004/0121952 A1
(43) Pub. Date: Jun. 24, 2004

(54) Treatment of cancer

(75) Inventors: Shmuel Ben-Sasson, Jerusalem (IL); Hadas Reuveni, Mevaseret Ziyyon (IL)

Correspondence Address: BROWDY AND NEIMARK, P.L.L.C., 624 NINTH STREET, NW, SUITE 300, WASHINGTON, DC, 20001-5303, US

(73) Assignees: Children's Medical Center Corporation, Boston, MA (US); Yissum Research and Development, Jerusalem (IL)

(21) Appl. No.: 10/455,787

(22) Filed: Jun. 6, 2003

Related U.S. Application Data
(63) Continuation-in-part of application No. 10/012,030, filed on Dec. 11, 2001.
(63) Continuation-in-part of application No. 08/861,153, filed on May 21, 1997, now Pat. No. 6,723,694.
Application No. 10/012,030 is a continuation-in-part of application No. 09/735,279, filed on Dec. 11, 2000, now abandoned.
Application No. 09/735,279 is a continuation-in-part of application No. 08/861,153, filed on May 21, 1997, now Pat. No. 6,723,694.
(60) Provisional application No. 60/385,900, filed on Jun. 6, 2002.

(51) Int. Cl. A61K 38/17; C07K 14/47

(52) U.S. Cl. 514/12; 530/350

(57) ABSTRACT

The present invention concerns methods for the treatment of solid tumors by the inhibition of Lyn associated signal transduction. Preferred in accordance with the invention are inhibitors which comprise sequences derived from specific regions of the Lyn. The present invention further concerns a method for the treatment of cancer by the administration of compounds comprising Lyn-derived peptides.

### CROSS-REFERENCE TO RELATED APPLICATIONS

The present application claims benefit of U.S. provisional application 60/385,900, filed Jun. 6, 2002, and is also a continuation-in-part of U.S. application Ser. No. 10/012,030, filed Dec. 11, 2001, and a continuation-in-part of U.S. application Ser. No. 08/861,153, filed May 21, 1997. Said application Ser. No. 10/012,030 is a continuation-in-part of U.S. application Ser. No. 09/735,279, filed Dec. 11, 2000, now abandoned, which is a continuation-in-part of said U.S. application Ser. No. 08/861,153, filed May 21, 1997. The entire context of each of the above applications is incorporated herein by reference.

### FIELD OF THE INVENTION

The present invention concerns methods and compositions for the treatment of cancers.

### BACKGROUND OF THE INVENTION

Protein tyrosine kinases are members of the eukaryotic protein kinase superfamily. Enzymes of this class specifically phosphorylate tyrosine residues of intracellular proteins and are important in mediating signal transduction in multicellular organisms. Protein tyrosine kinases occur as membrane-bound receptors, which participate in transmembrane signaling, or as intracellular proteins which take part in signal transduction within the cell, including signal transduction to the nucleus.

As such, phosphorylation of tyrosine residues by protein tyrosine kinases is an important mechanism for regulating intracellular events in response to environmental changes. A wide variety of cellular events, including cytokine responses, antigen-dependent immune responses, cellular transformation by RNA viruses, oncogenesis, regulation of the cell cycle and modification of cell morphology and phenotype are regulated by protein tyrosine kinases.

Enhanced protein tyrosine kinase activity can lead to persistent stimulation by secreted growth factors, for example, which, in turn, can lead to proliferative diseases such as cancer, to nonmalignant proliferative disease such as arteriosclerosis, psoriasis and to inflammatory response such as septic shock.

Src is among the first protein kinases described whose uncontrolled expression is directly linked to malignant transformation. The Src family contains several members. Some of these members are ubiquitously expressed while others like Lck, Hck, and Lyn have been, until recently, thought to be expressed primarily in cells of the immune system.

Lyn (also referred to at times as “Lyn tyrosine kinase”) is an intracellular-membrane associated tyrosine kinase expressed mainly in hematopoietic cells of myeloid and B-lymphoid origin. Lyn plays an indispensable role in the signaling mediated through thr B-cell antigen receptor and the high affinity receptor for FcE and has been implicated in signaling from members of the cytokine receptor super family such as I1-3, GM-CSF, and IL-5.

The term “carcinoma” refers to a malignant neoplasm of epithelial origin or cancer of the internal or external lining of the body. Carcinomas, malignancies of epithelial tissue, account for 80 to 90 percent of all cancer cases and are thus in the focus of drug development efforts.

Epithelial tissue is found throughout the body. It is present in the skin, as well as the covering and lining of organs and internal passageways, such as the gastrointestinal tract

Carcinomas are divided into two major subtypes: adenocarcinoma, which develops in an organ or gland, and squamous cell carcinoma, which originates in the squamous epithelium.

Adenocarcinomas generally occur in mucus membranes and are first seen as a thickened plaque-like white mucosa. They often spread easily through the soft tissue where they occur. Squamous cell carcinomas occur in many areas of the body.

Most carcinomas affect organs or glands capable of secretion, such as the breasts, which produce milk, or the lungs, which secrete mucus, or colon or prostate or bladder.

### SUMMARY OF THE INVENTION

The present invention is based on the surprising finding that short peptides, corresponding to short sequences from specific regions of Lyn, or variants of said sequences, were able to reduce growth of cancer cells of many different types both in vivo and in vitro, notably of solid tumors

The present invention is further based on the surprising finding that said short peptides were capable of inhibiting Lyn-associated signal transduction, as determined for example by the decrease in the phosphorylation level of Lyn kinases in their presence in a dose dependant manner, thus leading to the understanding that the inhibition of the Lyn-associated signal transduction (hereinafter “LAST”) leads to the reduction of growth of cells from solid tumors. While there has been publication linking the inhibition of Lyn to the treatment of leukemia a cancer of hematopoietic origin, there has been no disclosure showing that inhibition of Lyn is capable of improving solid tumors.

Thus, by a first aspect, the present invention concerns a method for the reduction of growth of cancer cells comprising: administering to the cells a compound comprising an amino acid sequence that corresponds to sequences in specific regions of Lyn (hereinafter: the “HJ-loop, B4-B5 region, αD region, A-region”) or to variants of said sequence.

The method of reduction of growth of cancer cells can be used as a therapeutic method for the treatment of cancer in an individual in need of such treatment.

The present invention further concerns methods for identifying the variants of said sequences effective in the reduction of growth of tumor cells. By a second aspect, the present invention concerns a method for reducing growth of cells from solid tumors by administration to the cells of at least one inhibitor of LAST.

The method may be used as a therapeutic method for the treatment of solid tumors.

The inhibitors of LAST may be compounds comprising amino acid sequences corresponding to sequences present in the above specific regions of the Lyn-kinase, or variants of said sequences; antisense sequences complementary to regions of the Lyn gene or Lyn mRNA, so that hybridization between the antisense and the Lyn-gene or Lyn mRNA can reduce protein expression; dominant negative Lyn that cause a decrease in activity of the native kinase; ribozymes capable of specifically cleaving Lyn RNA; anti-Lyn antibodies capable of entering cellular membranes or expression construct capable of transfecting cell which can express anti-Lyn antibodies, and small organic molecules capable of inhibiting LAST.

The most preferred inhibitors of the LAST, in accordance with the present invention, are compounds that comprise short amino acid sequences corresponding to sequences present in the above specific regions of a Lyn, or variants of said sequence. More preferably the region is the HJ-loop.

Without wishing to be bound by theory, it is assumed that the amino acid sequence, present in said compounds, mimics these specific regions in the Lyn, which are regions that interact with cellular components, such as with the substrates of the Lyn, phosphatases of the Lyn, Lyn itself (of the same or different molecule) that interact with the Lyn and possibly phosphorylate the kinase, with regulators of the kinase and the like. This mimic sequence is assumed to bind to the cellular components (for example to the substrates of Lyn) and this binding causes the interruption of the interaction of the Lyn with said cellular components (which may be a separate molecule or another region of the Lyn molecule itself. This interruption causes the inhibition of the signal transduction mediated by Lyn, thus leading to the reduction of the growth of cancer cells.

### GENERAL DESCRIPTION OF THE INVENTION

By one aspect, the present invention concerns a method for the reduction in the growth of cancer cells the method comprising:

contacting the cells with an effective amount of a compound comprising a sequence selected from:

(a) a sequence which is a continuous stretch of at least five amino acids present in Lyn in positions 434-458 (HJ loop);

(b) a sequence which is a continuous stretch of at least five amino acids present in Lyn in positions 318-336 (αD region);

(c) a sequence which is a continuous stretch of at least five amino acids present in Lyn in positions 305-316 (B4-B5 region);

(d) a sequence which is a continuous stretch of at least five amino acids present in a native Lyn in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequences of (a) to (d);

(f) a sequence of any one of (a) to (e) wherein at least one of the amino acids is replaced by the corresponding D-amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h).

The term “reduction of growth” refers to a decrease in at least one of the following: number of cells (due to cell death which may be necrotic, apoptotic or a combination of the above) as compared to control; decrease in growth rates of cells, i.e., the to total number of cells may increase but at a lower level or at a lower rate than the increase in control; decrease in the invasiveness of cells (as determined for example by soft agar assay) as compared to control even if their total number has not changed; and progression of non-differentiated cancer cells to a more differentiated phenotype.

The term “treatment of cancer” in the context of the present invention refers to at least one of the following: decrease in tumor size; decrease in rate of tumor growth; stasis of tumor size; decrease in the number of metastasis; decrease in the number of additional metastasis; decrease in invasiveness of the cancer; decrease in the rate of progression of the tumor from one stage to the next, as well as decrease in the angiogenesis induced by the cancer.

The term “compound (comprising sequence)” refers to a compound that includes within any of the sequences of (a) to (i) as defined above. The compound may be composed mainly from amino acid residues, and in that case the amino acid component of the compounds should comprise no more than a total of about 55 amino acids. Where the compound is mainly an amino acid compound, it may comprise of any one of the amino acid sequences of (a) to (h), a combination of two or more, preferably of three most preferably of two, of the sequences of (a) to (h) linked to each other (either directly or via a spacer moiety). The compound may further comprise any one of the amino acids sequences, or combinations as described above (in (a) to (i) above), together with additional amino acids or additional amino acid sequences. The additional amino acids may be sequences from other regions of the Lyn-kinase, for example sequences that are present in the kinase vicinity of the above regions (HJ loop, A-region, αD-region, B4-B5), N-terminal or C-terminal to the sequences of (a) to (d), or sequences which are not present in Lyn but were included in the compound in order to improve various physiological properties such as penetration into cells (sequences which enhance penetration through membranes or barriers which are generally termed “leader sequences”); decreased degradation or clearance; decreased repulsion by various cellular pumps, improved immunogenic activities, improvement in various modes of administration (such as attachment of various sequences which allow penetration through various barriers, through the gut, etc.); increased specificity, increased affinity, decreased toxicity, and the like. A specific example is the addition of the amino acid Gly, or of several Gly residues in tandem, to N-terminal of the sequence.

The compound may also comprise non-amino acid moieties, such as for example, hydrophobic moieties (various linear, branched, cyclic, polycyclic or heterocyclic hydrocarbons and hydrocarbon derivatives) attached to the peptides of (a) to (i) to improve penetration; various protecting groups, especially where the compound is linear, which are attached to the compound's terminals to decrease degradation. Chemical (non-amino acid) groups present in the compound may be included in order to improve various physiological properties such as penetration into cells (moieties which enhance penetration through membranes or barriers); decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity, for imaging purposes and the like. The chemical groups may serve as various spacers, placed for example, between one or more of the above amino acid sequences, so as to spatially position them in suitable orientation in respect of each other.

The compounds of the invention may be linear or cyclic, and cyclization may take place by any means known in the art. Where the compound is composed predominantly of amino acids/amino acid sequences, cyclization may N- to C-terminal, N-terminal to side chain and N-terminal to backbone, C-terminal to side chain, C-terminal to backbone, side chain to backbone and side chain to side chain, as well as backbone to backbone cyclization. Cyclization of the compound may also take place through the non-amino acid organic moieties.

The association between the amino acid sequence component of the compound and other components of the compound may be by covalent linking, by non-covalent complexion, for example, by complexion to a hydrophobic polymer, which can be degraded or cleaved producing a compound capable of sustained release; by entrapping the amino acid part of the compound in liposomes or micelles to produce the final compound of the invention. The association may be by the entrapment of the amino acid sequence within the other component (liposome, micelle) or the impregnation of the amino acid sequence within a polymer to produce the final compound of the invention.

Preferably the compounds comprise an amino acid sequence of (a) to (i) above in association with (in the meaning described above) a moiety for transport across cellular membranes.

The term “moiety for transport across cellular membranes” refers to a chemical entity, or a composition of matter (comprising several entities) that causes the transport of members associated (see above) with it through phospholipidic membranes. One example of such moieties are hydrophobic moieties such as linear, branched, cyclic, polycyclic or heterocyclic substituted or non-substituted hydrocarbons. Another example of such a moiety is short peptides that cause transport of molecules attached to them into the cell by, gradient derived, active, or facilitated transport. Other examples of other non-peptidic moieties known to be transported through membranes such as glycosylated steroid derivatives, are well known in the art. Yet another example is moieties that are endocytosed by cellular receptors such as ligands of the EGF and transferrin receptors. The moiety of the compound may be a polymer, liposome or micelle containing, entrapping or incorporating the amino acid sequence therein. In the above examples the compound of the invention is the polymer, liposome micelle etc. impregnated with the amino acid sequence.

The term “a sequence which is a continuous stretch of at least 5 amino acids present . . . ” means any continuous stretch of having a minimum of 5 amino acids to a maximum of the full length of the region, which are present within or is an amino acid sequence described by reference to positions of Lyn-kinase. For example, in the HJ-loop defined as positions 434-458 of Lyn, the continuous stretch of at least 5 amino acids may be from amino acid at position 434 to 438, from 435 to 439, from 436 to 440, . . . 444-458. The continuous sequence may also be of 5, 6 (435 to 440 . . . 453 to 456), 7, 8, 9, 10, 11, 12, 13, 14, 15, 16, 17, 18, 19 or 20 amino acids, obtained from each of these regions.

The term “Lyn” in reference to specific positions concerns protein tyrosine kinase denoted as EC 2.7.1.12, splice from A-human Accession TVHULY, PID g66782 (NCBI database).

The term “wherein up to 40% of amino acids of the native sequence have been replaced with a naturally or non-naturally occurring amino acid or with a peptidomimetic organic moiety” in accordance with the present invention, concerns an amino acid sequence, which shares at least 60% of its amino acid with the native sequence as described in (a), (b), (c) or (d) above, but some of the amino acids were replaced either by other naturally occurring amino acids, (both conservative and non-conservative substitutions), by non-naturally occurring amino acids (both conservative and non-conservative substitutions), or with organic moieties which serve either as true peptidomimetics (i.e., having the same steric and electrochemical properties as the replaced amino acid), or merely serve as spacers in lieu of an amino acid, so as to keep the spatial relations between the amino acid spanning this replaced amino acid. Guidelines for the determination of the replacements and substitutions are given in the detailed description part of the specification. Preferably no more than 30%, 25%, 20% or 10% of the amino acids are replaced.

The term “wherein up to 40% of the amino acids have their side chains chemically modified” refers to a variant which has the same type of amino acid residue, but to its side chain a functional group has been added. For example, the side chain may be phosphorylated, glycosylated, fatty acylated, acylated, iodinated or carboxyacylated. Other examples of chemical substitutions are known in the art and given below.

The term “up to 20% of the amino have been deleted” refers to an amino acid sequence which maintains at least 20% of its amino acid. Preferably no more than 10% of the amino acids are deleted and more preferably none of the amino acids are deleted.

The term “provided that at least 50% of the amino acids in the parent protein are maintained unaltered in the variants” the up to 40% substitution, up to 40% chemical modification and up to 20% deletions are combinatorial, i.e., the same variant may have substitutions, chemical modifications and deletions so long as at least 50% of the native amino acids are identical to those of the native sequence both as regards the nature of the amino acid residue and its position in the sequence. In addition, the properties of the parent sequence, in modulating Lyn-associate signal transduction, have to be maintained in the variant typically, at the same or higher level.

When calculating 40% (or 35, 30, 25, 20%) replacement of 20% (or 10%) deletion from sequences, the number of actual amino acids should be rounded mathematically, so that both 40% of an 11 mer sequence (4.4) and 40% of a 12 mer sequence (4.8) is five amino acids.

Typically “essential amino acids” are maintained or replaced by conservative substitutions while non-essential amino acids may be maintained, deleted or replaced by conservative or non-conservative replacements. Generally, essential amino acids are determined by various Structure-Activity-Relationship (SAR) techniques (for example amino acids when replaced by Ala cause loss of activity) are replaced by conservative substitution while non-essential amino acids can be deleted or replaced by any type of substitution. Guidelines for the determination of the deletions, replacements and substitutions are given in the Detailed Description Part of the specification.

The term “region” refers to a sequence in a specific location is the Lyn-kinase that corresponds to the positions selected from: 434 to 458 (termed: HJ loop); positions 318-336 (termed: αD region); position 305-313 (termed: B4-B5 region) and position 291-308 (termed: A-region).

The term “corresponding D-amino acid” refers to the replacement of the naturally occurring L-configuration of the natural amino acid residue by the D-configuration of the same residue.

The term “at least one peptidic backbone has been altered to a non-naturally occurring peptidic backbone” means that the bond between the N- of one amino acid residue to the C- of the next has been altered to non-naturally occurring bonds by reduction (to —CH₂—NH—), alkylation (methylation) on the nitrogen atom, or the bonds have been replaced by amidic bond, urea bonds, or sulfonamide bond, etheric bond (—CH₂—O—), thioetheric bond (—CH₂—S—), or to —CS—NH—; The side chain of the residue may be shifted to the backbone nitrogen to obtain N-alkylated-Gly (a peptidoid).

The term “in reverse order” refers to the fact that the sequence of (a) to (f) may have the order of the amino acids as it appears in the native Lyn from N- to the -C direction, or may have the reversed order (as read in the C- to N-direction) for example, if a subsequence of the HJ-loop of Lyn is GIVTYGK (SEQ ID NO:17) a sequence in a reverse order is KGYTVIG (SEQ ID NO:18). It has been found that many times sequences having such a reverse order can have the same properties, in small peptides, as the “correct” order, probably due to the fact that the side chains, and not the peptidic backbones are those responsible for interaction with other cellular components. Particularly preferred, are what is termed “retro inverse” peptides—i.e., peptides that have both a reverse order as explained above, and in addition each and every single one of the amino acids, has been replaced by the non-naturally occurring D-amino acid counterpart, so that the net end result, as regards the positioning of the side chains, (the combination of reverse order and the change from L to D) is zero change. Such retro-inverso peptides, while having similar binding properties to the native peptide, were found to be resistant to degradation.

The present invention further concerns a method for the treatment of cancer in a subject comprising administering to the subject, in need of such treatment, a therapeutically effective amount of a compound comprising a sequence selected from:

(a) a sequence which is a continuous stretch of at least five amino acids present in Lyn in positions 434-458 (HJ loop);

(b) a sequence which is a continuous stretch of at least five amino acids present in Lyn in positions 318-336 (αD region);

(c) a sequence which is a continuous stretch of at least five amino acids present in Lyn in positions 305-316 (B4-B5 region);

(d) a sequence which is a continuous stretch of at least five amino acids present in a native Lyn in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequence of (a) to (d);

(f) a sequence of any one of (a) to (e) wherein at least one of the amino acids is replaced by the corresponding D-amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h).

The present invention also concerns use of a compound comprising a sequence selected from:

(a) a sequence which is a continuous stretch of at least five amino acids present in Lyn in positions 434-458 (HJ loop);

(b) a sequence which is a continuous stretch of at least five amino acids present in Lyn in positions 318-336 (αD region);

(c) a sequence which is a continuous stretch of at least five amino acids present in Lyn in positions 305-316 (B4-B5 region);

(d) a sequence which is a continuous stretch of at least five amino acids present in a native Lyn in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant and provided that the variant maintains the biological activity of the parent sequence of (a) to (d);

(f) a sequence of any one of (a) to (e) wherein at least one of the amino acids is replaced by the corresponding D-amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h);

for the preparation of a medicament for the treatment of cancer.

The term “treatment of cancer” includes at least one of the following: decrease in the rate of growth of the cancer (i.e., the cancer still grows but at a slower rate); cease of growth of the cancer growth, i.e., stasis of the tumor growth, and, in preferred cases, the tumor diminishes or is reduced in size. The term also concerns reduction in the number of metastasis, reduction in the number of new metastasis formed, slowing of the progression of the cancer from one stage to the other and decrease in angiogenesis induced by the cancer. In most preferred cases, the tumor is totally eliminated. This term also concern prevention for prophylactic situations or for those individuals who are susceptible to contracting tumor, the administration of said compounds will reduce the likelihood of the individual contrasting the disease. In preferred situations, the individual to whom the compound is administered does not contract the disease.

The term “cancer” in the context of the present invention includes all types of neoplasm whether in the form of solid or non-solid tumors, from all origins, and include both malignant and benign conditions as well as their metastasis. In particular this term refers to: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, cancer of the thyroid, head and neck cancer, brain cancer, cancer of the peripheral nervous system, cancer of the central nervous system, neuroblastoma, cancer of the endometrium, myeloid lymphoma, leukemia, acute myelocytic leukemia, chronic leukemia, Hodgkin's lymphoma, non-Hodgkin's lymphoma as well as metastasis of all the above.

The term “solid tumors” refers to carcinomas, sarcomas, adenomas, and cancers of neuronal origin and if fact to any type of cancer which does not originate from the hematopoietic cells and in particular concerns: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above.

The present invention also concerns a method for obtaining of the most favorable compounds comprising the above sequences (a) to (i), for the reduction in the growth of cancer cells.

Thus the present invention concerns a method for obtaining compounds for the treatment of cancer, the method comprising:

(a) identifying peptide regions in Lyn that are in positions selected from: 434-458 (HJ-loop), 291-308 (A-region), 305-313 (B4-B5 region), 318-336 (αD region);

(b) synthesizing a plurality of compounds comprising a sequence selected from:

(b1) a sequence corresponding to at least five continuous amino acid sequences of the HJ-loop, A-region, B4-B5 or αD region;

(b2) a variant of the sequence according to (b 1) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequence of (a) to (d);

(b3) a sequence of (b 1) or (b2) wherein one or more of the amino acids has been replaced by the corresponding D-amino acid;

(b4) a sequence of (b1), (b2) or (b3) wherein at least one of the peptidic backbone has been altered to a non-naturally occurring amino acid;

(b5) a sequence being the sequence of any one of (b1), (b2), (b3) or (b4) in a reverse order; and

(b6) a combination of two or more sequences of (b1)-(b5);

(c) testing the modulation activity of the compounds of (b) in a test assay for determining their activity in the reduction of growth of cancer cells;

(d) selecting from the compounds of (c) those compounds which caused reduction of the growth of said cells in the test assay as compared to the reduction of growth in the same test assay in the absence of the compound; and

(e) producing the compounds of (d) thereby obtaining compounds for the reduction of growth of cancer cells.

Preferably, the amino acid sequence of (a) above should be in positions 434 to 458 of Lyn (a region defined as the HJ-loop), more preferably in positions selected from: 436 to 441 of the Lyn, 441-453 of the Lyn or 447-456 of the Lyn (sub-regions of the HJ-loop).

The amount of compounds of the invention administered to the individual will depend on the type and severity of the disease (for example the stage of the cancer) and on the characteristics of the individual, such as general health, age, body weight and tolerance to drugs as well as on the mode of administration. The skilled artisan will be able to determine appropriate dosages depending on these and other factors. Typically, a therapeutically effective amount of the compound can range from about 1 mg per day to about 1000 mg per day for an adult. Preferably, the dosage ranges from about 1 mg per day to about 100 mg per day.

By a second aspect the present invention concerns a method for reduction of growth of cancer cells from solid tumors comprising administering to the cancer cells an effective amount of a LAST-inhibitor.

The invention concerns methods for the treatment of solid tumors comprising administering to a subject, in need of such treatment, a therapeutically effective amount of an inhibitor of LAST.

Any inhibitor of LAST can be administered to the individuals in the course of treating solid tumors. Among the Lyn-tyrosine kinase inhibitors that can be employed are compounds comprising sequences derived from Lyn regions responsible for interaction with cellular components or variants of such sequences as described above, antibodies immunoreactive with Lyn for example antibodies prepared by gene therapy techniques or antibodies present in compounds of formulations (liposomes) that can penetrate cellular membranes, anti-sense nucleic acids that block expression of Lyn; dominant negative Lyn genes which express Lyn proteins with reduced or non-existent biological activity, ribozymes that specifically cleave Lyn RNA and small organic molecules that inhibit Lyn. Any of these inhibitors of Lyn will inhibit the growth of solid tumors in individuals.

The term “solid tumors” in the context of the present invention concerns carcinomas, sarcomas, adenomas and any type of cancer which is not from the hematopoietic origin.

Preferably the LAST inhibitors are compounds comprising sequences derived from regions of the Lyn that are responsible for interaction with cellular components, especially with the Lyn-substrates or with other or the same kinase molecules. As indicated above, it is assumed that peptides mimicking said regions, binds to the cellular components (such as substrates of the Lyn, phosphatases, kinase regulators, other kinase-molecules, or other regions of the same kinase molecules), and by this interrupt the interaction of the Lyn and the substrate, leading to inhibition of LAST.

More specifically, the LAST inhibitor is a compound comprising a sequence selected from:

(i) a compound comprising a sequence selected from:

(a) a sequence which is a continuous stretch of at least five amino acids present in Lyn in positions 434-458 (HJ loop);

(b) a sequence which is a continuous stretch of at least five amino acids present in Lyn in positions 318-336 (αD region);

(c) a sequence which is a continuous stretch of at least five amino acids present in Lyn in positions 305-316 (B4-B5 region);

(d) a sequence which is a continuous stretch of at least five amino acids present in Lyn in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);

(f) a sequence of any one of (a) to (e) wherein at least one of the amino acids is replaced by the corresponding D-amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h);

(ii) a compound comprising an antibody, or antigen-binding portion thereof, reactive with Lyn wherein said compound is capable of penetrating through cellular membranes; or an expression construct capable of expressing said antibody;

(iii) an antisense nucleic acid sequences complementary to a region in the Lyn gene or Lyn mRNA, so that hybridization between said antisense and said gene or hybridization between said antisense and said RNA, results in decrease in expression of Lyn;

(iv) a small interfering RNA (siRNA) being complementary or identical to a region in the Lyn mRNA so that hybridization of said siRNA and the Lyn mRNA results in degradation of the Lyn mRNA;

(v) a ribozyme that specifically cleaves Lyn RNA;

(vi) an expression constructs coding for dominant negative Lyn; and

(vii) small organic molecules capable of inhibiting Lyn.

More specifically the sequence of (I) is selected from any one of the sequences disclosed in FIG. 1A (disclosing HJ-loop derived sequences) as well as sequences present in FIG. 1B disclosing HJ-full sequence; HJ-subsequence; αD-region-full sequence; αD-subsequence; B4-B5-full sequence A-region-full sequence.

This invention also relates to the reduction of the growth of cells from solid tumors by administering one or more inhibitors of LAST to the cells. The administration of inhibitors of LAST to the cells causes a reduction in the growth of these cells and, at least eventually, causes a reduction in the number of these cells. Again, any inhibitor of LAST will inhibit the growth of cells from solid tumors when delivered to these cells. The inhibitors include the compounds comprising the peptides from the regions defined above and their variants, antibodies (capable of entering the cellular membrane), anti-sense nucleic acids, negative dominant LAST genes, and small organic molecules.

The term “Lyn-associated signal transduction (LAST)” refers to the level of signaling mediated by Lyn, which is best evaluated by determination of the phosphorylation level of at least one substrate in the Lyn-signaling pathway which may be a direct substrate of Lyn (Lyn itself, CD19, CD79, Vav, Syk, Shc, PI3-kinase (p85), N-Myristoyltransferase (NMT), FAK, Protein Band 3, Syk, SLP-65, Tec protein tyrosine kinase, HSI)) or a substrate of another kinase more downstream in the Lyn signaling pathway, such as MAP kinase, ERK, JNK, or P13K dependent-kinase, PDK and PKB.

The sequences which correspond to regions of Lyn, in addition to their ability to reduce the growth of solid tumors in individuals or their ability to inhibit the growth of cells from solid tumors, also are useful for generating antibodies that reduce the growth solid tumors and inhibit the growth of cells obtained from solid tumors. The sequences act as antigenic agents for producing such antibodies. These antibodies, in turn, act as inhibitors of LAST, thereby reducing the growth of solid tumors and inhibiting growth of cells from solid tumors when they are administered to the individual

### BRIEF DESCRIPTION OF THE DRAWINGS

FIG. 1A shows sequences (SEQ ID Nos:1-11) illustrating the amino acid sequences of the HJ-loop-derived peptides including modified sequences; and FIG. 1b additional sequences (SEQ ID Nos:12-16) from the HJ-loop and other regions of Lyn.

FIGS. 2A to 2K are a graphs showing the percent inhibition of proliferation, as compared to control, of various cells lines, MCF7 (human breast cancer), MDA231 (human breast cancer), 1063 (ovary cancer), HEC-1A (endometrium cancer), HS703T (colon cancer), Colo205 (colon cancer), EMT (breast cancer mouse), C6 (glioma) NIC H727 (in AMI 47) and NIC H727 in AMI 159-both being lung cancer, 293 cells kidney epithelial, respectively by increasing concentrations of compounds of the invention K055H302; K055H719, and K055H101 (see FIG. 1A for sequences).

FIG. 3 Western blots showing the Lyn expression in various cancer cell lines.

FIG. 4 shows phosphorylation levels of several Lyn substrates, determined by co-immunoprecipitation in the presence of varying concentrations of the compound of the invention K055H302 (see FIG. 1A for sequences).

FIG. 5 shows interruption of the interaction between Lyn and its substrates in the presence of the compound of the invention.

FIG. 6 shows histochemical staining with Lyn-antibodies in sections obtained from prostate, colorectal, and urinary bladder cancer patients.

FIG. 7 shows northern blot of Lyn mRNA expression in prostate cancer DU-145 cells, the presence and absence of siRNA Lyn duplexes, after 24 and 48 hour incubation. Actin mRNA serving as control.

FIG. 8 shows inhibition of prostate cancer cells (DU145) proliferation in the presence of Lyn siRNA, and in the presence of a non-relevant siRNA.

FIG. 9 shows western blots of indicating inhibition of phosphorylation of ERK, a Lyn substrate, in the presence and absence of siRNA Lyn.

FIG. 10 shows in vivo results of tumor shrinkage in a xenograph model of ovarian cancer in the presence of the compound of the invention K055H302.

### DETAILED DESCRIPTION OF THE INVENTION

By one aspect the present invention is directed to the inhibition of cancer cells (of any type) by administration of novel compounds which comprise peptides derived from specific regions of the Lyn, or variants of these sequences.

By another aspect he present invention is based on the finding that Lyn-tyrosine kinase is active in many cell lines obtained from solid tumors and that the reduction of the signal transduction associated with the kinase (as determined for example by the reduction of the phosphorylation of the kinase substrates) inhibits the growth of these cells. Thus the administration of inhibitors of LAST, causes the inhibition of the signal transduction leading to the reduction in the proliferation of cells from solid tumors.

This invention is also directed to methods for inhibiting the growth of cells from solid tumors, whether within the body of an individual, in the originating tissue of the tumor or in metastasis, or anywhere outside an individual's body, such as in an in vitro setting. These methods are directed to administering one or more inhibitors of LAST to the cells from solid tumors. The inhibitor or inhibitors is (are) administered in amounts that are effective in reducing the growth of the tumor cells. When the inhibitors are administered to the cells, the cells stop proliferating (growing or dividing) as rapidly as they did in the absence of the inhibitors. In many instances, growth of the cells from solid tumors entirely ceases for example since the cells lose their viability and die. The growth retardation, or death of the cells from the solid tumors occurs because Lyn tyrosine kinase associated signal transduction is involved with growth and viability of these cells.

Any inhibitor of LAST will thus serve to decrease the level of Lyn-associated signal transduction and thus will act to decrease growth of cancer cells. as will be explained below.

Small Molecule Inhibitors

Low molecular weight organic molecules can act as inhibitors of Lyn tyrosine kinase directly (by binding) and by this inhibit the LAST. Such low molecular weight organic molecules are known in the art. Exemplary of such compounds is the pyrazolone pyrimidine tyrosine kinase inhibitor PP1, or PP2 (see Schindler et al. “Crystal Structure of Hck in Complex with a Src Family-Selective Tyrosine Kinase Inhibitor”, Molecular Cell, Vol. 3, 639-648, May 1999, the pertinent contents of which are incorporated herein by reference. Other organic compound inhibitors of the Src family tyrosine kinases are known. Preferred low molecular weight organic molecules for use with the present invention are those that specifically inhibit the activity of Lyn.

Ribozymes That Specifically Cleave Lyn-RNA

A specific modulator of LAST is a ribozyme that is a catalytic oligonucleotide (typically RNA). The catalytic oligonucleotide can be tailored to specifically recognize, via hybridization, a specific mRNA region and thus cleave it and eliminate its expression. The ribozymes may be introduced to the cell as catalytic RNA molecules or as expression constructs for the expression of the catalytic RNA molecules.

Antisense LAST Inhibitors

Another type of inhibitor of LAST is anti-sense nucleic acids. The nucleic acids are single stranded ribonucleic or deoxyribonucleic acid strands which contain nucleotides joined together through normal sugar-phosphate bonds. Antisense sequences can inhibit production of Lyn-protein by one of three mechanisms. By a first mechanism these antisense interfere with transcription as these antisense hybridize within the structural gene or in the regulatory gene thereof, that encodes for Lyn tyrosine kinase. This hybridization interrupts the transcription of Lyn gene into mRNA.

A second mechanism is the binding of the antisense in the cytoplasm to the Lyn mRNA, thus interfering with the formation of a proper translation construct leading to inhibition of translation of the protein. This leads to the decrease in the amount of Lyn-protein produced and thus to an inhibition of LAST.

A third mechanism is the formation of a double-stranded mRNA-antisense duplex which leads to rapid degradation of mRNA duplex by RNases (such as RNase H). All these mechanisms lead to production of smaller amounts of Lyn-produced by the cells than without the presence of these anti-sense nucleic acids, thus leading to LAST inhibition.

The particular nucleotides that are joined together to form the anti-sense sequence are those that are complementary to a region of the Lyn tyrosine kinase structural gene, or complementary to regulatory region of the gene sufficient to inhibit production of functional Lyn. These nucleotides of the anti-sense nucleic acids are specifically determined by the nucleotides of the target location and can easily be identified by the skilled practitioner once the sequence of the target location is established. The target location is a matter of choice to some extent. It lies within the region of the structural gene that encodes Lyn tyrosine kinase or in the regulatory coding region of the structure. The target location nucleotide sequence can easily be established by the skilled practitioner from publicly available information concerning the Lyn tyrosine kinase gene or can be obtained by routine examination of homologous genes coupled with standard molecular biology techniques.

By one option, the antisense is an oligonucleotide of several to several tens of nucleotides that are inserted into the cells. This is the preferred oligonucleotide in accordance with the invention. Typically the sequence is the first 20-25 nucleotides in the 5′ terminal of the Lyn cDNA (that are complementary to the mRNA). An example of such sequence is

5′ atggga tgtataaaat caaaagggaa agac (SEQ ID NO:19),

or an RNA sequence as the above, wherein t has been replaced by u

Another option is the use of longer antisense sequences (up to several hundred nucleotides) by insertion into an expression vector, which can then transfected into the tumor cell by various gene transfer technologies. If that case the full sequence of the Lyn can be used to construct a sequence which is complementary to it to produce a long antisense mRNA complementary to the native RNA. Finding the target of the kinase sequence to be used for antisense purposes may be carried out by screening through various overlapping sequences, or by use of various bioinformative software that can locate likely targets in a given gene and give several alternative sequences for producing antisense sequences that can eliminate production.

Small Interference RNA (siRNA) Inhibitors of LAST

Yet another option of inhibiting Lyn-expression is by inhibiting the translation of the mRNA to protein by the use of small interference RNS (siRNA). RNA interference (RNAi) is a recently discovered phenomenon, whereby, double-stranded RNA (dsRNA) is introduced into a cell, leading ultimately to the degradation of messenger RNA (mRNA) containing an identical or complementary sequence, effectively silencing the targeted gene. Small interfering RNA (siRNA) is a 21-23 nucleotide long RNA that mediates messenger RNA (mRNA) catalysis. RNAi is the mechanism of sequence-specific, post-transcriptional gene silencing initiated by double-stranded RNAs (dsRNA) homologous to the gene being suppressed. dsRNAs are processed by Dicer, a cellular ribonuclease III, to generate duplexes of about 21 nt with 3′-overhangs (small interfering RNA, siRNA) which mediate sequence-specific mRNA degradation. In mammalian cells siRNA molecules are capable of specifically silencing gene expression without induction of the unspecific interferon response pathway.

Dominate Negative Kinase Genes

Still another type of inhibitor of LAST is negative dominant Lyn tyrosine kinase genes. The presence of these genes in the tumor cells allows non-functional Lyn tyrosine kinase to be expressed to the exclusion of functional Lyn tyrosine kinase, for example since both compete for binding to the substrate while the dominant negative kinase does not phosphorylate it. Dominant negative Lyn genes are introduced into tumor cells by gene transfer techniques, which are becoming increasingly more standard in the art (calcium precipitation, electrical discharge, physical injection, use of carriers such as recombinant vectors, etc.). The introduced dominant negative Lyn gene is incorporated in the cell's genome. There, copies of it are passed to progeny cells. Since this Lyn gene is dominant negative, it will be expressed in response to signals which induce Lyn tyrosine kinase expression rather than the active form of Lyn tyrosine kinase. Cells from solid tumors which have incorporated the dominant negative Lyn gene will not grow because the expressed Lyn is inactive. The dominant negative Lyn to genes can be found in the art or can be produced by standard gene mutation techniques which are well known to skilled practitioners in the art. These genes can be suitably packaged for transgenic procedures by appropriate methods and materials known to the skilled practitioners.

A specific example of such a gene is a sequence wherein the codon Lys 425 (AAA) in the region of the catalytic core encoding Lyn responsible to ATP-binding has been replaced with the Alanine or methione. Other examples are replacement of the codon of Lys 275(AAA) by codon for Arg (CGU/C/A/G www.pnas.org/cgi/content/full/98/18/10172 or replacement of the codon for Tyr 397 (TAC) by codon for the Phe

(aaa/c http:/emboj.oupjournals.org/cgi/content/full/6/7/1610.

Antibodies Against Lyn for Inhibitor LAST:

A further type of inhibition of LAST is antibodies that are immunoreactive with Lyn and are expressed in the cell as a results of gene transfection. These antibodies bind to the kinase and thereby severely limit or prohibit its kinase activity or interrupt its interaction with other cellular components, all the above leading to LAST inhibition. The antibodies can be of any class or type. The binding site of the antibodies can be anywhere on the Lyn molecule provided the immunoreactive binding between the antibody and the kinase molecule results in a severe inhibition of LAST. The antibodies are produced by transfecting the cell with an expression construct capable of expressing antibodies whether single or double chain. Alternatively, t antibodies or suitable binding fragments can be introduced into the cells by any of a variety of techniques known to the skilled practitioner (physical injection, attachment to carriers that cross cell membranes, transgenic introduction into the cells of the solid tumors for subsequent induction of expression, etc.). The secreted, introduced or expressed antibodies or suitable antibody fragments thereof immunoreactively bind to the Lyn-tyrosine kinase molecules, thereby inhibiting their activity. Commercially available anti-Lyn antibodies are available (Anti-Lyn (Santa Cruz, US) SC15 (44)).

Compounds Comprising Lyn Derived Peptides:

Many types of cancer, in accordance with the present invention, not only those derived from solid tumors may be reduced in growth by the administration of compounds comprising Lyn-derived peptides. These compounds comprising or consisting of said Lyn-derived peptides are the preferred inhibitors of LAST, in accordance with the invention and thus are the preferred agents for the reduction of growth of cancer and for the treatment of cancer in an individual. The peptides apparently mimic a region in the kinase and thus bind to other cellular components with which the Lyn interacts (such as the kinase substrates, other or the same molecules of the kinase and the like). This binding interrupts the kinase-component interaction (especially kinase-substrate interaction) and thus inhibit LAST. It should be noted that Lyn may form dimmers with other Lyn or may interact with other regions in its own molecule so the interruption may be of inter- or intra-Lyn interaction.

The compounds of the invention may cause reduction in the growth of cancer cells and may be used to treat cancer in an individual. Quite often the tumors are reduced in size and many times are eliminated altogether.

The peptides according to the above non-limiting theory mimic a region in the Lyn that is involved in the interaction of the Lyn with cellular components that are part of the Lyn-associated signal transduction. Preferably, these cellular components are selected from: the substrates of Lyn, other kinases (which may be other Lyn for trans- or auto-phosphorylation, or kinases of the same or different family), phosphatases, as well as regulators and ATP. Thus, any peptide which mimics a part of the Lyn responsible for said interaction can bind to the cellular component, and thus inhibit the LAST.

Specific preferred regions of the Lyn that the Lyn-derived peptides mimic are: the HJ-loop, αD-loop, A-region, and B4-B5 region, as defined above. It is clear that for interruption of the kinase-cellular component interaction there is no need to obtain a mimic of the full region of the kinase and a mimic of a subsequence may be sufficient to interrupt said interaction. It is further clear that the interruption may be caused by mimicking of any one of several smaller subsequences in the region and there is no necessity to mimic only one subsequence. It is further clear that for mimicking purposes it is not necessarily to obtain a sequence as present in the native kinase and variants of that sequence, that can faithfully copy the three dimensional structure of the region (when present in the full kinase), as well as copying the chemical characteristics of those side chains that bind to the substrate can also be used as mimics for interruption of the interaction. At times such variants may have better mimicking properties than the native sequence as the variation may help stabilize the mimic amino acid in a more favorable conformation.

The peptide derivative are short subsequences of at least five continuous amino acids obtained from the above sequences, as well as variants of the above sequences obtained by substitution of up to 40% of the amino acid with natural and non natural amino acids or with peptidomimetic moieties, and/or chemical modification of up to 40% of the amino acid residue, and/or deletions of up to 20% of the amino acids, provided that the peptide derivative has at least 50% of the amino acids as in the native peptide.

Most preferably, the sequence is at least five continuous amino acids obtained from the region of positions 434 to 458 HJ-loop, more preferably in positions 436 to 441 in said HJ-loop. The amino acid sequence may be a 5, 6, 7, 8, 9, 10, 11, 12, 13, 14, 15, 16 17 18 19 and 20 amino acids. The sequence may be the sequence of a naturally appearing in the HJ-loop. However, actual empirical experiments show that sequences having substitutions at times have better LAST inhibiting properties than native sequences. Therefore, in the scope of the present invention also includes variants of the native sequence of the at least five continuous amino acids from the region, in which up to 40% of the amino acids has been substituted, and/or up to 40% have been chemically modified, and/or up to 20% have been deleted. In general, amino acids in the regions, and in particular the HJ-loop region, which are essential for LAST, should be either identical to those appearing in the native sequence, chemically modified or substituted by conservative substitutions (in the context of the present invention the term “conservative substitutions” also refer to substitutions of charged amino acids by polar or hydrophobic amino acids having the same steric properties as will be explained bellow).

The other positions in the sequence may be replaced by conservative, non-conservative substitutions both by naturally and non naturally occurring amino acids as well as by organic peptidomimetics.

Preferably, Gly in position should be replaced by a D-amino acid, most preferably D-Lys, or D-Arg.

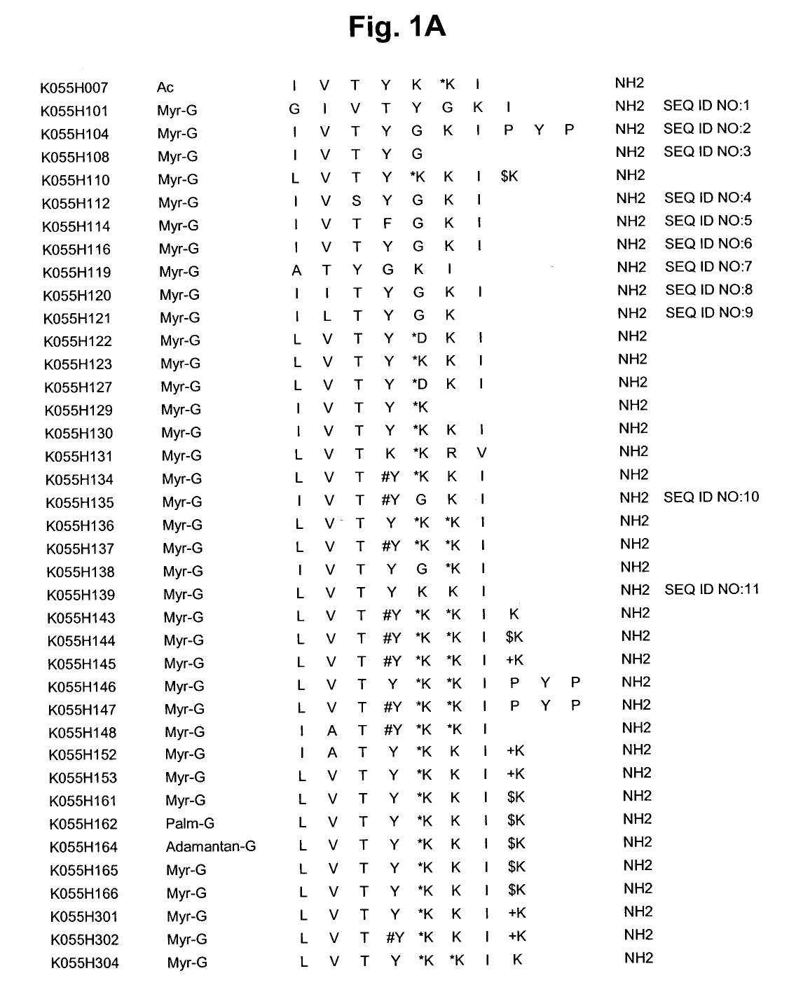

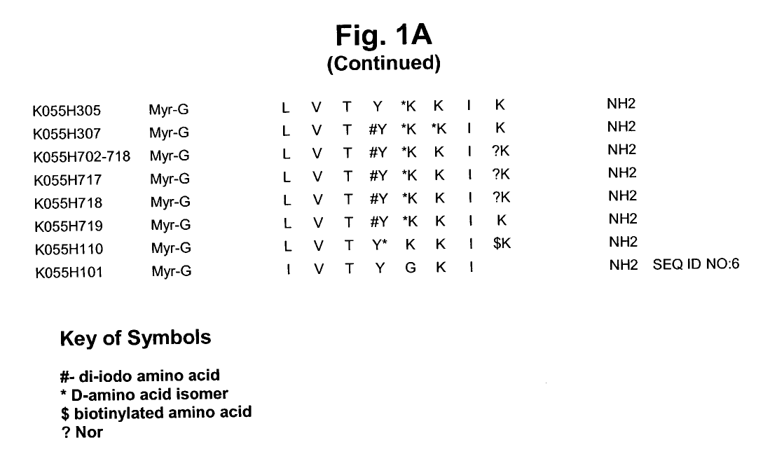

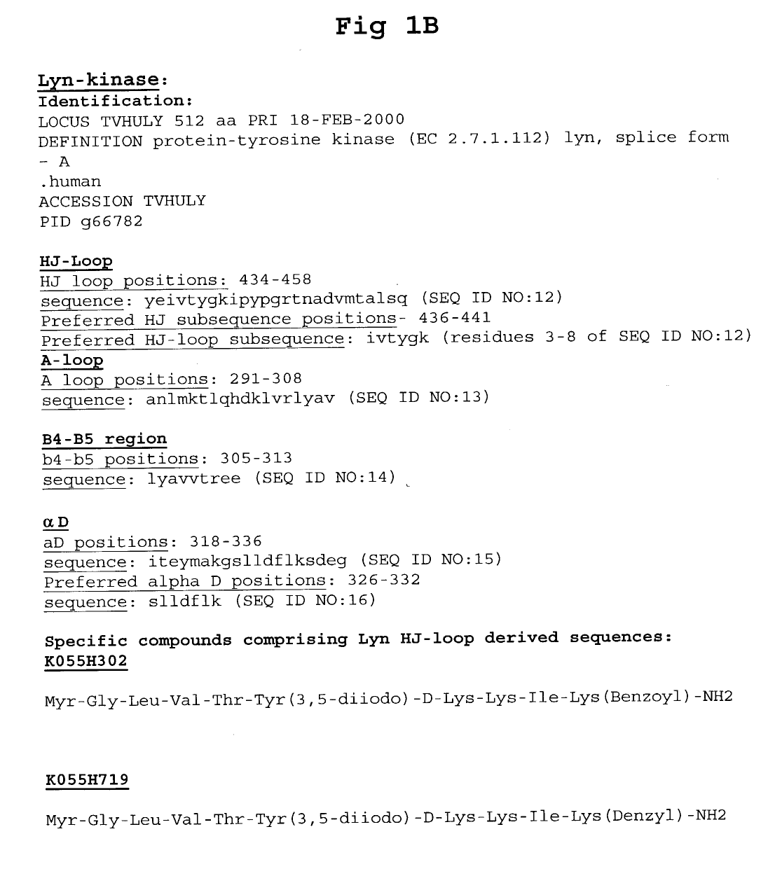

In this invention, particularly preferred compounds for inhibition of LAST are those specified in FIG. 1A or FIG. 1B and most preferably the compound specified in FIGS. 1a and 1B as K055H302.

Also included are peptides of FIG. 1A or 1B wherein 2-4 amino acids have been substituted, and/or 2-4 amino acids have been chemically substituted, preferably according to the guidelines given below.

1. Addition of Non-Peptidic Groups to one or to Both of the Terminals of the Sequences of (a) to (h) to Produce the Compound of the Invention Comprising a Lyn-Derived Peptide

Where the compound of the invention is a linear molecule, it is possible to place in any of its terminals various functional groups. The purpose of such a functional group may be for the improvement of the LAST inhibition. The functional groups may also serve for the purpose of improving physiological properties of the compound not related directly to LAST inhibition such as: improvement in stability, penetration (through cellular membranes or barriers), tissue localization, efficacy, decreased clearance, decreased toxicity, improved selectivity, improved resistance to repletion by cellular pumps, and the like. For convenience sake the free N-terminal of one of the sequences contained in the compounds of the invention will be termed as the N-terminal of the compound, and the free C-terminal of the sequence will be considered as the C-terminal of the compound (these terms being used for convenience sake). Either the C-terminus or the N-terminus of the sequences, or both, can be linked to a carboxylic acid functional groups or an amine functional group, respectively.

Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds these being an example for “a moiety for transport across cellular membranes”.

These moieties can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al, Biochem. Pharm. 37:3867 (1988), Anderson et al., Arch. Biochem. Biophys. 239:538 (1985) and Singhal et al., FASEB J 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a compound of the present invention is protected, preferably with a methyl, ethyl, benzyl or substituted benzyl ester, more preferably as a benzyl ester.

In addition, a modified lysine residue can be added to the C-terminal of the compound to enhance biological activity. Examples of lysine modification include the addition of an aromatic substitute, such as benzoyl benzoic acid, dansyl-lysine various derivatives of benzoic acids (difluoro-, trifluromethy-, acetamido-, dimethyl-, dimethylamino-, methoxy-) or various derivatives of carboxylic acid (pyrazine-, thiophene-, pyridine-, indole-, naphthalene-, biphenyl,), or an aliphatic group, such as acyl, or a myristic or stearic acid, at the epsilon amino group of the lysine residue.

Examples of N-terminal protecting groups include acyl groups (—CO—R₁) and alkoxy carbonyl or aryloxy carbonyl groups (—CO—O—R₁), wherein R₁ is an aliphatic, substituted aliphatic, benzyl, substituted benzyl, aromatic or a substituted aromatic group. Specific examples of acyl groups include acetyl, (ethyl)-CO-n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO— Examples of alkoxy carbonyl and aryloxy carbonyl groups include CH₃—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoyl, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH 2, —NHR₂ and —NR₂R₃) or ester (i.e., the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Preferably the compounds includes in the N-terminal a hydrocarbon having a length of C₄-C₂₀ preferably C₆-C₁₈, most preferably C₈-C₁₆. Example of hydrophobic moieties are: aaystyl, stearyl, lauroyl, palmitoyl and acetyl etc. Other examples are gernyl-gernyl, acetyl.

2. Finding a Shorter Subsequences of Lyn-Derived Peptides

As indicated, Lyn-derived peptides included in the compounds of the invention, whether used to inhibit cancer growth in general or used for inhibition of LAST, are obtained by finding which subsequence from the above regions (HJ-loop, A-region, αD-region, B4-B5 region) that caused reduction in the growth of cancer cells. Typically it is desired, for ease of synthesis and improved administration, to find the shortest sequence possible which is still active. In the following, the finding of the shortest sequence will be disclosed in connection with HJ-loop, but this description is applicable also to the other regions.

A shorter subsequence of the HJ-loop comprising a continuous stretch of at least five amino acid can be found by preparing a series of partially overlapping peptides each of 5-10 amino acids and each obtained by synthesizing a sequence that is one position removed from the previous sequence.

For example, the HJ-loop is in position 434 to 458, and it is to be desired to prepare 10 aa peptides, then the following, partially overlapping peptides are prepared, a peptide having the sequence 434-443, 435-444, 436-445 . . . 447-458. The cancer growth inhibiting activities of the subsequences is then determined in a test assay. The best 10-aa peptide is then chosen.

For checking whether the 10 aa peptide can be reduced in sequence, it is possible to either repeat the above procedure (preparing a series of partially overlapping peptides) using 5 aa long peptides that span the length of the chosen 10 aa peptide, or to shorten the 10 aa peptide by deleting alternatively from each terminal, an amino acid, and testing the cancer growth inhibiting activities activity of the progressively truncated peptides, until the optimal sequence of at least 5, at least 6, at least 7, at least 8, at least 9 aa peptide is obtained or until it is determined that longer sequences are required for maintaining this activity. As the HJ-loop (as well as the other regions) is relatively small, typically the number of different peptides to be tested is also small. For example, for an HJ-loop having a length of about 20 aa, there is a need to prepare only 12 peptides to find the optimal 8 aa peptide. After the best 8-aa peptide is obtained, it is possible to delete sequentially amino acids from one or both terminals of the 8 per peptide for obtaining the shortest sequence of 5, 6 or 7 aa that is still active. For these steps only 16 sequences have to be tested, so that by testing only 24 peptides it is possible to find such a shorter sequence having cancer growth inhibiting properties.

3. Identifying Essential and Non-Essential Amino Acids in the Subsequence Chosen

A. Ala-Scan

Once the shorter continuous stretch of at least 5 (at least 6, 7, 8, 9, 10, 11 or 12) amino acids has been identified, as explained above, it is necessary to realize which of the amino acids in the stretch are essential (i.e., crucial for the kinase-associated signal transduction modulation) and which are non-essential. Without wishing to be bound by theory, in almost every native protein involved in interaction with other cellular components, some amino acids are involved in the interaction (essential amino acids) and some amino acids are not involved in the interaction (non-essential amino acids), for example since they are cryptic. A short peptide which is to mimic a region of the Lyn protein behaves in the same way as the region when present in the full kinase: some amino acids actually interact with the substrate (or other interacting components) and other amino acids merely serve to spatially position the interacting amino acids, but do not participate in the interaction with the other cellular components.

Essential amino acids have to be maintained (i.e., be identical to those appearing in the native kinase), chemically modified or replaced by conservative substitutions (see definition below) to obtain variants of the peptides. Non-essential amino acids can be maintained, deleted, replaced by a spacer or replaced by conservative or non-conservative substitutions.

Identification of essential vs. non-essential amino acids in the peptide can be achieved by preparing several peptides that have a shorter sequence than the full region (see 2 above) in which each amino acid is sequentially replaced by the amino acid Ala (“Ala-Scan.”), or sequentially each amino acid is omitted (“omission-scan”). This allows to identify the amino acids which modulating activity is decreased by said replacement/omission (“essential”) and which are not decreased by said replacement/omission (“non-essential”) (Morrison et al., Chemical Biology 5:302-307, 2001). Another option for testing the importance of various peptides is by the use of site-directed mutagenesis. Other Structure-Activity-Relationship techniques may also be used.

B. 3D-Analysis

Another strategy for finding essential vs. non-essential amino acids is by determining which aa of the region, in the 3D of the full kinase, are exposed and which are cryptic. This can be done using standard software such as SPDB viewer, “color by accessibility” of Glaxo-Welcome.

Typically cryptic aa are non-essential and exposed or partially exposed amino acids are more likely to be essential. However, if one wishes to “guess” theoretically which “non-conservative” substitutions in the cryptic region can be tolerated, a good guideline is to “check” on a 3D computer model of the full kinase, whether a peptide superimposed on the full kinase and bearing those changes has still the overall structure of the region and more importantly, whether the exposed amino acids in the variants still overlap the positions of the exposed amino acids in the full kinase. Those non-conservative substitution, that when simulated on a computer's 3D structure (for example using the Triphose™ software) do not cause drastic alteration of the overall shape of the region (drastic shifting in the position of the exposed amino acids) are likely non-conservative replacements. Thus prior to experimental testing it is possible to reduce the number of tested candidates by computer simulation. Where the 3D structure of a specific kinase is not available in activating crystallography data, it is possible to obtain a “virtual” 3D structure of the kinase based on homology to known crystallographic structures using such progress such as CompSer™ (Tripose, USA).

4. Obtaining Variants

The sequence regions of the compound of the invention may be the native sequences obtained from Lyn (preferably the shortest possible sequence from the region that has the highest activity), or alternatively variants of the native sequence obtained by deletion, (of non-essential amino acids) or substitution (only conservative substitutions in essential positions, both conservative and non-conservative of non-essential acids) or chemical modification.

4.1 Deletions and Insertions

Deletions can occur in particular of the “non-essential amino acids”. Additions may occur in particular at the N-terminal or the C-terminal of any of the amino acids of the sequence. No more than 20%, preferably 10% most preferably none of the amino acids should be deleted. Insertions should preferably be N-terminal or C-terminal to the sequence of (a) to (h) or between the several sequences linked to each other in (i). However other insertions or deletions are possible. Again, the feasibility of the deletions in creating a peptide which is a good mimic can be evaluated virtually by reverting to the 3D-module as described above, and finding which deletions still maintain the exposed side chains (when the peptide is superimposed on the kinase in the same positions).

4.2 Replacements

The variants can be obtained by replacement (termed also in the text as “substitution”) of any of the amino acids as present in the native kinase. As may be appreciated there are positions in the sequence that are more tolerant to substitutions than others, and in fact some substitutions may improve the activity of the native sequence. The determination of the positions may be realized using “Ala-Scan,” “omission scan” “site directed mutagenesis” or 3-D theoretical considerations as described in 3 above. Generally speaking the amino acids which were found to be “essential” should either be identical to the amino acids present in the native specific kinase or alternatively substituted by “conservative substitutions” (see bellow). The amino acids that were found to be “non-essential” might be identical to those in the native peptide, may be substituted by conservative or non-conservative substitutions, and may be deleted or replaced by a “spacers”.

The term “naturally occurring amino acid” refers to a moiety found within a peptide and is represented by —NH—CHR—CO—, wherein R is the side chain of a naturally occurring amino acid.

The term “non-naturally occurring amino acid” (amino acid analog) is either a peptidomimetic, or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. This term also refers to the D-amino acid counterpart of naturally occurring amino acids. Amino acid analogs are well known in the art; a large number of these analogs are commercially available. Many times the use of non-naturally occurring amino acids in the peptide has the advantage that the peptide is more resistant to degradation by enzymes which fail to recognize them.

The term “conservative substitution” in the context of the present invention refers to the replacement of an amino acid present in the native sequence in the specific kinase with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid). However where the native amino acid to be replaced is charged, the conservative substitution according to the definition of the invention may be with a naturally occurring amino acid, a non-naturally occurring amino acid or a peptidomimetic moiety which are charged, or with non-charged (polar, hydrophobic) amino acids that have the same steric properties as the side-chains of the replaced amino acids. The purpose of such a procedure of maintaining the steric properties but decreasing the charge is to decrease the total charge of the compound, for example for improving its membrane penetrating properties.

For example in accordance with the invention the following substitutions are considered as conservative: replacement of arginine by cytroline; arginine by glutamine; aspartate by asparagine; glutamate by glutamine.

As the naturally occurring amino acids are grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The following are some non-limiting examples of groups of naturally occurring amino acids or of amino acid analogs are listed bellow. Replacement of one member in the group by another member of the group will be considered herein as conservative substitutions:

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes glycine, alanine, valine, serine, cysteine, threonine and a modified amino acid having an ethyl side chain. Preferably Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, μ-hydroxyarginine, N-amidinocitruline and 2-amino-4-guanidinobutanoic acid, homologs of lysine, homologs of arginine and ornithine. Preferably, Group V includes histidine, lysine, arginine, and ornithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

The term “non-conservative substitutions” concerns replacement of the amino acid as present in the native Lyn by another naturally or non-naturally occurring amino acid, having different electrochemical and/or steric properties, for example as determined by the fact the replacing amino acid is not in the same group as the replaced amino acid of the native kinase sequence. Those non-conservative substitutions which fall under the scope of the present invention are those which still constitute a compound having kinase-associated signal transduction modulating activities. Because D-amino acids have hydrogen at a position identical to the glycine hydrogen side-chain, D-amino acids or their analogs can often be substituted for glycine residues, and are a preferred non-conservative substitution

A “non-conservative substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH₂)₅—COOH]—CO— for aspartic acid.

Alternatively, a functional group may be added to the side chain, deleted from the side chain or exchanged with another functional group. Examples of non-conservative substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄ COOH for the side chain of serine. These examples are not meant to be limiting.

As indicated above the non-conservative substitutions should be of the “non-essential” amino acids.

Preferably, the Lyn may be substituted by benzylamine groups, by biotinylation. Another substitution is di-iodinization of tyrosine. Any amino acid may be replaced by its D-isomer and in particular Lys may be replaced by its D-isomer.

“Peptidomimetic organic moiety” can be substituted for amino acid residues in the compounds of this invention both as conservative and as non-conservative substitutions. These peptidomimetic organic moieties either replace amino acid residues of essential and non-essential amino acids or act as spacer groups within the peptides in lieu of deleted amino acids (of non-essential amino acids). The peptidomimetic organic moieties often have steric, electronic or configurational properties similar to the replaced amino acid and such peptidomimetics are used to replace amino acids in the essential positions, and are considered conservative substitutions. However such similarities are not necessarily required. The only restriction on the use of peptidomimetics is that the compounds retain their tissue-remodeling modulating activity as compared to compounds constituting sequence regions identical to those appearing in the native kinase.

Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988));

LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al, J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

4.3 Chemical Modifications

In the present invention the side amino acid residues appearing in the native sequence may be chemically modified, i.e., changed by addition of functional groups. The modification may be in the process of Lyn-synthesis of the molecule, i.e., during elongation of the amino acid chain and amino acid, i.e., a chemically modified amino acid is added. However, chemical modification of an amino acid when it is present in the molecule or sequence (“in situ” modification) is also possible.

The amino acid of any of the sequence regions of the molecule can be modified (in the peptide conceptionally viewed as “chemically modified”) by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

4.4 Cyclization of the Molecule

The present invention also includes cyclic compounds which are cyclic molecules.

A “cyclic molecule” refers, in one instance, to a compound of the invention in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the sequence present therein, preferably the side chain of the C-terminal amino acid. For example, an amide can be formed between the nitrogen atom at the N-terminus and the carbonyl carbon in the side chain of an aspartic acid or a glutamic acid. Alternatively, the compound can be cyclized by forming a covalent bond between the carbonyl at the C-terminus of the compound and the side chain of a suitable amino acid in the sequence contained therein, preferably the side chain of the N-terminal amino acid. For example, an amide can be formed between the carbonyl carbon at the C-terminus and the amino nitrogen atom in the side chain of a lysine or an ornithine. Additionally, the compound can be cyclized by forming an ester between the carbonyl carbon at the C-terminus and the hydroxyloxygen atom in the side chain of a serine or a threonine.

“Cyclized” also refers to forming a ring by a covalent bond between the side chains of two suitable amino acids in the sequence present in the compound, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a compound can be cyclized with a linking group between the two termini, between one terminus and the side chain of an amino acid in the compound, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or omithine, for example) or acetamidomethyl (Acm) (for the sulfhydryl of cysteine) protecting groups. OAI and Aloc are easily removed by Pd and Acm is easily removed by iodine treatment.

5. Pharmaceutical Compositions and Therapeutical Methods of Treatment

The inhibitor of LAST of the present invention or the compounds for reduction of cancer cell growth can be used as active ingredients (together with a pharmaceutically acceptable carrier) to produce a pharmaceutical composition. The pharmaceutical composition may comprise one, or a mixture of two or more of the different LAST inhibitors of the invention in an acceptable carrier or may comprise one or more compounds comprising Lyn-derived peptides.

The pharmaceutical composition comprising the Lyn-derived peptides can be used for the treatment of any type of cancer. The pharmaceutical compositions comprising the LAST inhibitors can be used for the treatment of any solid tumor such as for example: carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium as well as for the treatment of metastasis of any of the above.

The LAST inhibitors of the present invention can be administered parenterally. Parenteral administration can include, for example, systemic administration, such as by intramuscular, intravenous, subcutaneous, or intraperitoneal injection. Compounds which resist proteolysis can be administered orally, for example, in capsules, suspensions or tablets. The compound can also be administered by inhalation or insufflations or via a nasal spray.

The compounds of the invention can be administered to the individual in conjunction with an acceptable pharmaceutical carrier as part of a pharmaceutical composition for treating the diseases discussed above. Suitable pharmaceutical carriers may contain inert ingredients which do not interact with the compounds. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release ofBiological Active Agents, John Wiley and Sons, 1986). The formation may be also resources for administration to bone, or in the form of salve, solution, ointment, etc. for topical administration.

The pharmaceutical compositions may also be administered in conjunction with other modes of therapy (chemotherapy, radiotherapy) routinely used in the treatment of cancer.

A “therapeutically effective amount” is the quantity of compound which results in an improved clinical outcome as a result of the treatment compared with a typical clinical outcome in the absence of the treatment An “improved clinical outcome” results in the individual with the disease experiencing fewer symptoms or complications of the disease, including a longer life expectancy, as a result of the treatment. With respect to cancer, an “improved clinical outcome” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

6. Determination of LAST Inhibiting Activity

It should be appreciated that some of the compounds that comprise sequences (a)-(i) above are better LAST inhibitors than others and/or some of the compounds are better than others in reduction of growth of cells from solid tumors. Some of the conservative substitutions in the essential positions may diminish the inhibiting activities, while other such conservative substitution in the essential positions may improve these inhibiting activities. The same is true also for deletions, substitutions (both conservative and non-conservative) in non-essential positions, as well as to chemical modifications (in any position) or insertions. In addition the type and size of the non-amino acid portion of the compounds, such as a hydrophobic moiety in one of its terminals may diminish or increase the LAST inhibiting activities. The LAST inhibiting activities that can be determined for example by using one of the assays stipulated below.

6.1 Cellular Assays

It can be readily determined whether a compound modulates the activity of a LAST by incubating the compound with cells which have one or more cellular activities controlled by the LAST. Examples of these cellular activities include cell proliferation, cell differentiation, cell morphology, cell survival or apoptosis, cell response to external stimuli, gene expression, lipid metabolism, glycogen or glucose metabolism and mitosis. The cells are incubated with the candidate compound to produce a test mixture under conditions suitable for assessing the level of the LAST. The activity of the LAST is assessed and compared with a suitable control, e.g., the activity of the same cells incubated under the same conditions in the absence of the candidate compound (or in the presence of a control compound). A lesser activity of LAST in the test mixture compared with the control indicates that the candidate compound inhibits LAST.

Suitable cells for the assay include normal cells which express Lyn (such as B-cells), cells which have been genetically engineered to express a Lyn, malignant cells expressing a Lyn or immortalized cells that express the kinase.

Conditions suitable for assessing activity include conditions suitable for assessing a cellular activity or function under control of the LAST pathway. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors or of specific activators such as cytokines, hormones and the like).

For example, the proliferation of transformed cell—may be determined as in Example 2 below, i.e., determination of proliferation (for example as determined by methylene-blue dye assay).

Another cellular assay is for determining the change of invasiveness of tumor (by using a soft agar assay, as specified in Examples 4 below.

6.2 Phosphorylation of Substrates (in Cellular or Cell Free Assays)

It is possible to assess the LAST activity and the changes in this LAST as compared to control, by determining the phosphorylation level of the substrate proteins of the Lyn. Examples of possible Lyn substrates are: (Lyn itself, CD19, CD79, Vav, Syk, Shc, P13-kinase (p85), N-Myristoyltransferase (NMT), FAK, Protein Band 3, Syk, SLP-65, Tec protein tyrosine kinase, HSI). Cells known to express the Lyn such as for example B-lymphocytes are incubated with a candidate compound for inhibiting the LAST and are activated by addition of ant-IgM ligands. Then the cells are lysed, the protein content of the cells is obtained and separated on a gel. The substrates can be identified by use of suitable molecular weight markers, or by using suitable antibodies, reactive against Lyn, CD19, CD79, Syk, Vav, PI3 kinase (p85), Shc, etc. The level of phosphorylation of the substrate may be determined by suing labeled anti-Tyr antibodies. Alternatively, the suitable substrate may be immuno-precipitated using antibodies. The level of substrate phosphorylation in the immuno-precipitate can be determined by using anti-phosphotyrosine antibodies (see Fujimoto et al., Immunity, 13:47-57 (2000)).

By another option, phosphorylation may be determined in a cell-free system by incubating a mixture comprising Lyn, the substrate of the kinase and candidate molecules for inhibiting LAST in the presence of ATP under conditions enabling phosphorylation. The proteins are then subjected to gel separation, transferred to nitrocellulose where the substrate band is identified by antibody or molecular weight marker followed by immunoblotting by anti-phosphotyrosine antibody. Alternatively it is possible to use [γ-³² P] ATP and quantify the amount of radioactivity incorporated in the substrate (See Fujimoto et al., The J. of Immol. 7088-7094 (1999). Assays concerning phosphorylation of substances can be seen in Example 5.

6.3. Tissue or In Vivo Assay

Suitable assays for determining inhibition of LAST can be by inducing prostate tumor in an experimental animal, by implanting subcutaneously cell lines obtained from tumors (such as for example MCF7 (human breast cancer), MDA231 (human breast cancer), 1063 (ovary cancer), HEC-1A (endometrium cancer), HS703T (colon cancer), Colo205 (colon cancer), EMT (breast cancer mouse), C6 (glioma) NIC H72747 (lung cancer) in an experimental animal such as nude mice and then testing the effect of the candidate compound on one of the following: tumor size (decease in size, stasis or decreased growth rates as compared to control), progression of tumor to advanced stages (determined by histological techniques), survival of animals, spread of metastasis, angiogenesis and the like. Such an assay is shown in Example 3 below.

7. Preparation of Antibodies

The Lyn-derived peptides of the present invention can be useful in the preparation of specific antibodies against Lyn tyrosine kinase. Suitable antibodies can be raised against a Lyn peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al., Nature, 256:495-497 (1975) and Eur. J. Immunol. 6:511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

The antibodies can be used to determine if an intracellular Lyn tyrosine kinase is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-Lyn peptide antibody specific for Lyn. The lysate is then analyzed, for example, by Western blotting or immunoprecipitation for complexes between Lyn tyrosine kinase and antibody. Anti-Lyn peptide antibodies can be utilized for the study of the intracellular distribution (compartmentalization) of Lyn tyrosine kinase under various physiological conditions via the application of conventional immunocytochemistry such as immunofluorescence, immunoperoxidase technique and immunoelectron microscopy, in conjunction with the specific anti-Lyn peptide antibody.

Antibodies reactive with the Lyn peptides are also useful to detect and/or quantity the Lyn in a sample, or to purify the Lyn (e.g., by immunoaffinity purification).

The Lyn-derived peptides of the present invention can also be used to identify ligands which interact with Lyn and which inhibit the activity of Lyn. For example, an affinity column can be prepared to which a Lyn peptide is covalently attached, directly or via a linker. This column, in turn, can be utilized for the isolation and identification of specific ligands which bind the Lyn peptide and which will also likely bind the Lyn tyrosine kinase. The ligand can then be eluted from the column, characterized and tested for its ability to inhibit Lyn function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5:315 (1992)). The teachings of these references are incorporated herein by reference.

Methods of cyclizing compounds having peptide sequences are described, for example, in Lobl et al, WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

8. Preparation of the Compounds

Peptide sequences for producing any of the sequence of the compounds of the invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Aarifield, J. Am. Chem. Soc., 88:2149 (1963); Meienhofer, Hormonal Proteins and Peptides, C. H. Li, Ed., Academic Press, 1983, pp. 48-267; and Barany and Aarifield, in The Peptides, E. Gross and J. Meienhofer, Eds., Academic Press, New York, 1980, pp. 3-285. Methods of solid phase peptide synthesis are described in Aarifield, R. B., Science, 232:341 (1986); Carpino, L. A. and Han, G. Y., J. Org. Chem., 37:3404 (1972); and Gauspohl, H. et al., Synthesis, 5:315 (1992)). The teachings of these references are incorporated herein by reference.

As indicated above the compounds of the invention may be prepared utilizing various peptidic cyclizing techniques. Methods of cyclizing compounds having peptide sequences are described, for example, in Lobl et al., WO 92/00995, the teachings of which are incorporated herein by reference. Cyclized molecules can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or omithine, for example) or acetamidomethyl (Acm) (for the sulfhydryl of cysteine) protecting groups. OAI and Aloc are easily removed by Pd and Acm is easily removed by iodine treatment.

Other modes of cyclization (beyond N- to C-terminal cyclization) may include: N- to backbone cyclization, C- to backbone cyclization, N- to side chain cyclization, C- to side chain cyclization, backbone to side chain cyclization, backbone to backbone cyclization and side chain to side chain cyclization.

### EXAMPLE 1

### Preparation of Compounds Comprising Lyn-Derived Peptides

The compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J. Org. Chem., 37: 3404 (1972); Gauspohl, H., et al., Synthesis, 5: 315 (1992)). The teachings of which are incorporated herein by reference.

Rink Amide Resin [4(2′,4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzo-triazolyl-1-yl)-1,1,3,3-tetramethyluronium) dissolved in HOBt (1-hydroxy-benzotriazole) and DMF (dimethylformamide). The amino acid was dissolved in this solution with additional NMP. DIEA (diisopropylethylamine) was added to initiate activation. Alternatively, the activation method of DCC (dicycbohexylcarbodiimide) and HOBL was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisoie; 0.5 ml D.I. H₂O; 10 ml TFA.

### EXAMPLE 2

### Inhibition of Proliferation of Tumor Cells Obtained From by Incubation with Compounds Comprising Lyn-Derived Peptides

The following Human solid tumors cell lines: MCF7 (human breast cancer), EMT ((mouse breast cancer), MDA231 (human breast cancer), 1063 (ovary cancer), HEC-1A (endometrium cancer) HS703T (colon cancer), Colo205 (colon cancer) EMT (breast cancer mouse), all dissolved in formulation MiriB (see bellow) C6 (glioma) NIC H727 (dissolved in AMI 47) and NIC H727(dissolved in AMI 159-both being lung cancer, 293 cells (kidney epithelial cells) were obtained from the American Type Culture Collection. These cell lines were grown in RPMI 1640 medium supplemented with penicillin (100 U/ml), streptomycin (100 μg/ml), glutamine (2 mM) and 10% endotoxin free bovine cell serum (Hyclone).

A suspension of the cells at 2×10⁴ cells/ml was prepared in the above described culture mediums and distributed 0.180 ml per well (about 4000 cells/well) in the wells of 96 well, flat bottom, tissue culture microtiter plates.

A series of compounds stock solutions were prepared by diluting a 10 mM solution of the compound in 100% DMSO with phosphate buffered saline (PBS) containing 0.1% bovine serum albumin (BSA) to a concentration of 400 μM. These solutions were labeled DMSO. In many instances, 40 μl of the 10 compound in DMSO solution was mixed with 160 μl of 2M NH₄HCO₃ and heated for 40 minutes at 100° C. The resultant solution was then diluted to 400 μM in PBS containing 0.1% BSA. These compounds stock solutions were labeled “tbi”. The concentration of compound in each stock solution was adjusted to nine times the desired concentration of the compound in the assay mixture. 0.020 ml of each compound stock solution was added to the corresponding wells about 2 hours after cell addition, with six replicates for each concentration. In addition, PBS containing 0.1% BSA solution with no added compound was used as a control. The plates were incubated for 72-80 hours at 37° C. in a 10% CO₂ humidified incubator. This formulation was termed “tbi”, and served as a vehicle and as control.

The plates were labeled and the medium discarded. The wells were fixed with 4% formaldehyde PBS (PBS buffered with 10% formalin from Fisher Scientific; Catalog No. HC200-1) (0.2 ml/well) for at least 30 minutes. The wells were washed one time with borate buffer (0.2 ml/well) (0.1M, pH 8.5). Freshly filtered 1% methylene blue solution (0.60 ml/well) was then added to the wells and incubated for 10 minutes at room temperature. The wells were then washed five times with tap water, after which the wells were dried completely. 0.20 mUwell of 0.1 N HCl was added to extract the color. After overnight extraction, the O.D. was read at 630 nm to determine the number of cells per well. The procedure for counting cells is described in greater detail in Oliver et al. J. Cell Sci., 92: 513 (1989), the teachings of which are incorporated herein by reference.

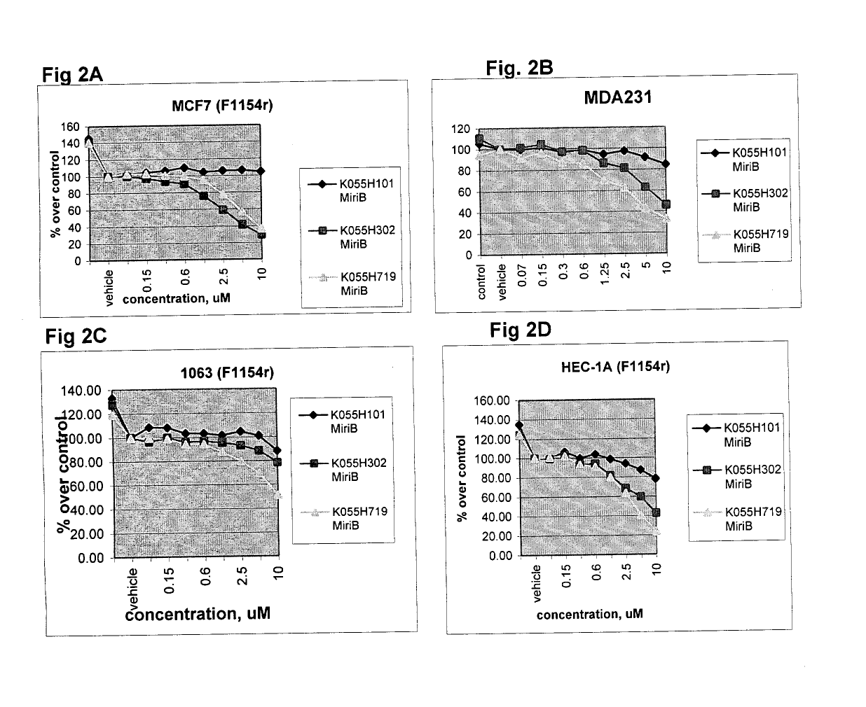

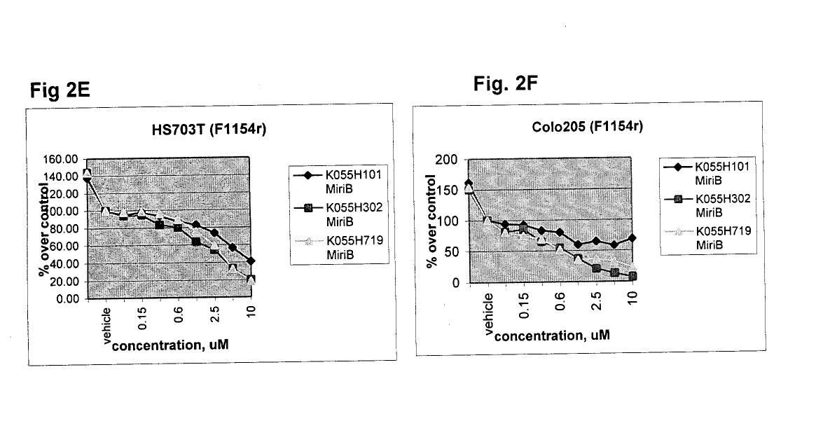

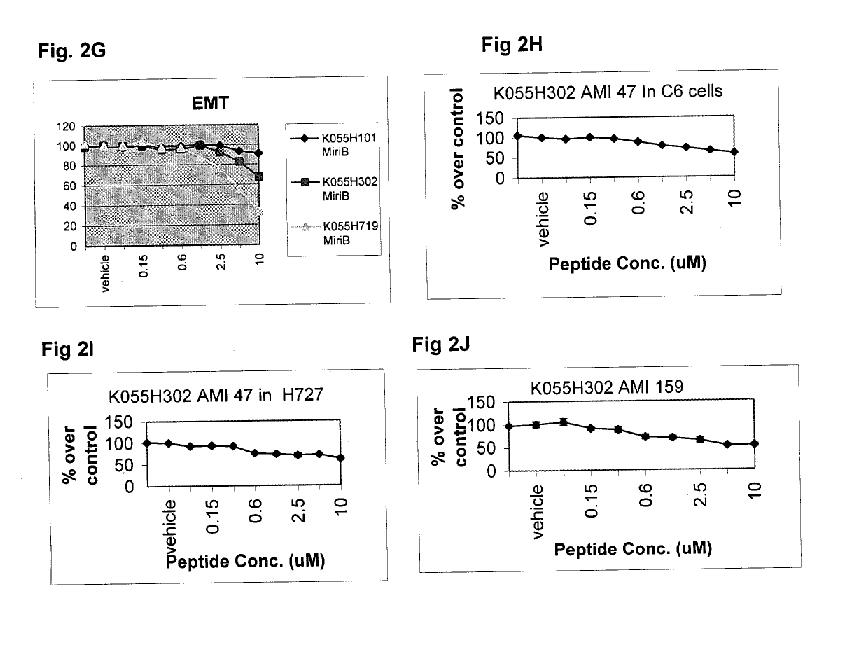

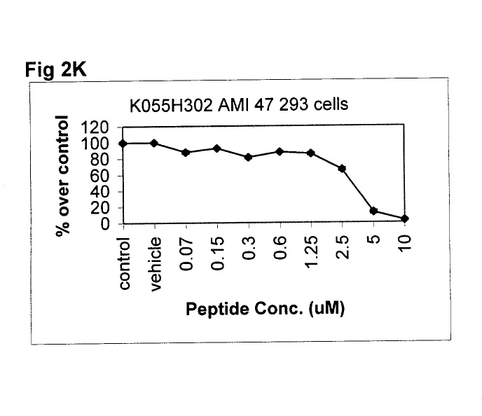

The results are shown in FIGS. 2A to 2K. (2H, 2I a 2J, and 2K include results obtained only from K055H30). The data in these figures show that compounds, comprising Lyn derived peptides K055H101 (corresponding to the native sequence of the HJ-loop); K055H302; K055H719 which are both sequences modified as compared to the native sequence we were able to inhibit growth of several different lines of cancer cells in a dose dependant manner.

It should be noted that, as a rule, the peptides K055h302 and k055719 showed which have modified sequences as compared to control feature better cancer growth inhibiting activities than the native sequence K055H101.

### EXAMPLE 3

### Preparation Formulations

3A: B-blac Formulation

15 mg of the compound were dissolved in 0.25 ml of 4% benzyl alcohol, 4% Pluronic L44 (BASF, Mount Olive, N.J.) and 2% benzyl benzoate in propylene glycol. To this, 0.125 ml of 2.2% glycine in DDW and 0.125 ml of 50 mM sodium bicarbonate were added while vigorously stirring the tube. The preparation was heated to 100° C. for 15 min., then homogenized with Polytron (Kinematica, Luzan, Switzerland) for 2′ during which 0.5 ml of 0.3 M lactose were gradually added.

The sequence of heating and homogenizing was repeated once again and after that the preparation was sterilized by heating to 100° C. for 30 min.

3B: MiriB Formulation:

10 mg compound were dissolved in 0.5 ml of 4% benzyl alcohol and 4% Pluronic PE6200 (BASF, Mount Olive, N.J.) in propylene glycol. To this, 0.25 ml of 2.2% glycine in DDW and 0.25 ml of 50 mM sodium bicarbonate buffer (pH=7.5) were added while vigorously stirring the tube.

The preparation was homogenized with Polytron (Kinematica, Luzan, Switzerland) for 2 min., and then heated to 100° C. for 30′. The sequence of homogenizing and heating was repeated once again.

AMI 47—Protocol:

Resulting concentration is 10 mg/ml

| | | | % of total |
|---|---|---|---|
| Stage | Procedure | Solution Content | formulation |
| I | Dissolve 10 mg peptide in | 4% Benzyl Alcohol | 25% |
| | 0.250 ml solution, Vortex | 4% Pluronic L44 | |
| | | 2% Benzyl | |
| | | Benzoate | |
| | | in Propylene Glycol | |
| II | Add 0.125 ml of acidic | 2.2% Glycine in | 12.5% |
| | solution, Vortex | TDW (pH 5-5.5) | |
| III | Heat to 100° for 15′ | | |
| IV | Add 0.125 ml | 50 mM bicarbonate | 12.5% |
| | Neutralizing solution, | | |
| | Vortex | | |
| V | Add 0.250 ml isotonic | 5% Manitol in | 50% |
| | solution, Vortex. | TDW | |
| VI | Polytron: 20,000 rpm for 2′ | | |
| VII | Heat to 100° for 15′ | | |
| VIII | Polytron: 20,000 rpm for 2′ | | |
| IX | Heat to 100° for 30′ | | |

AMI 159—Protocol:

Resulting concentration is 10 mg 1 ml

| | | | % of total |
|---|---|---|---|
| Stage | Procedure | Solution Content | formulation |
| I | Dissolve 10 mg peptide in | 0.2% acetic acid in | 40% |
| | 0.40 ml acetic acid | 5% Mannitol in | |
| | solution, Vortex | TDW | |
| II | Add 0.150 ml PG | 4% Benzyl Alcohol | 15% |
| | solution, Vortex | 4% Pluronic L44 | |
| | | 2% Benzyl | |
| | | Benzoate | |
| | | in Propylene Glycol | |
| III | Heat to 100° for 15′ | | |
| IV | Neutralization 1 | 0.15 ml 100 mM | 15% |
| | | phosphate buffer | |
| | | pH 6.5 | |
| V | Neutralization 2 | 0.30 ml 100 mM | 30% |
| | | phosphate buffer | |
| | | pH 6.5 | |
| VI | Polytron: 20,000 rpm for 2′ | | |
| VII | Heat to 100° for 15′ | | |
| VIII | Polytron: 20,000 rpm for 2′ | | |
| IX | Heat to 100° for 30′ | | |

### EXAMPLE 4

### Change of Phosphorylation of Substrates

Experimental: cell lymphocytes cell line WEHI-231 was used. 5×10⁶ WEHI-231 cells/sample were washed with serum-free RPMI media (cells were spun at 1700 rpm for 5 min. at 4° C.). The cells were suspended in serum-free RPMI media at 2×10⁷ cells/ml, and lysed by addition of an equal volume cold 2×LB (80 mM Tris pH 7.5, 2% NP-40, 1% DOC, 0.2 SDS, 50 mM NaPPi, 100 mM NaF, 2 mM Na₃VO₄, protease inhibitor mix) on ice for 15 min. The resulting mixture was spun for 20 min. 17,000 rpm at 4° C. and supernatantly the cell extract was saved.

Immuno-precipitation (IP) of each target-protein was done in one batch: to the cell extract 2 μg of appropriate Ab/reaction were added and then cells were rotated o/n on at 4° C. 30 μl 50% of slurry sample of protein A/G beads (prewashed 3 times with cold 1×LB) were again added for 3 hr at 4° C. The IP complex was washed (×2) with cold 1×LB and (×2) with cold reaction buffer (50 mM Tris pH 7.5, 10 mM MgCl₂, 0.1 mM Na₃VO₄, 1 mM DTT). The resulting mixture was spun for 1 min. 14,000 rpm at 4° C. and each IP batch was divided into separate tubes.

For the kinase assay: appropriate volumes of the compound of K055H302 (see FIG. 1A) was added, or a control compound comprising an irrelevant sequence (obtained from a different kinase which is GRK) or a control of vehicle alone were added to each sample, and incubated for 20 min. at 30° C. Then 10 μM ATP and 5 units exogenous Lyn were added and incubated for 20 min. at 30° C. The reaction was stopped by addition of 8 μl 5×SDS sample buffer and boiled for 5 min. at 100° C. The resulting samples were separated on SDS-PAGE and blotted.

Western blot analysis was carried out with anti-phosphotyrosine, followed by stripping and rehybridization with the relevant antibodies.

The antibodies used in various assays:

| | |
|---|---|
| A-pTyr: | Upstate Biotechnology catalog # 05-321 |
| A-CD19: | Pharmingen catalog # 09651D |
| A-Lyn: | Santa Cruz sc-15 (44) |
| A-Syk: | Santa Cruz sc-1077 (N-19) |
| A-Vav: | Santa Cruz sc-132 (C-14). |

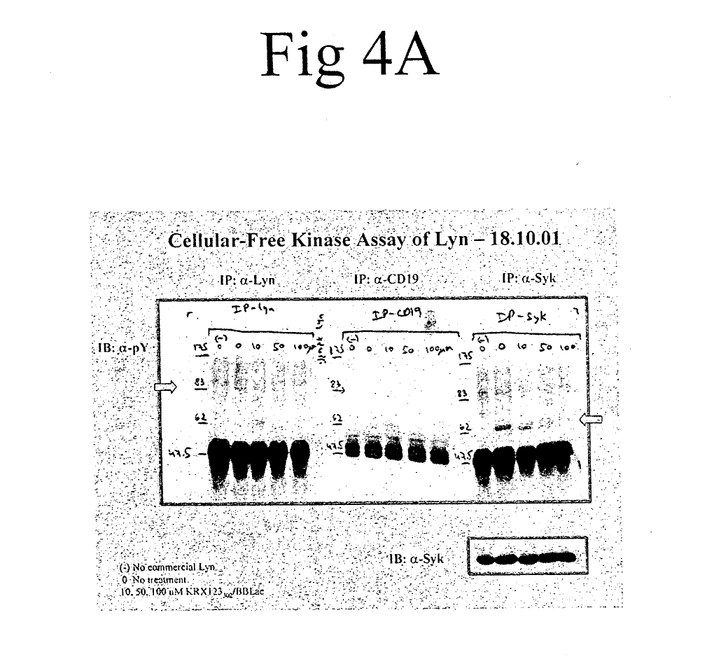

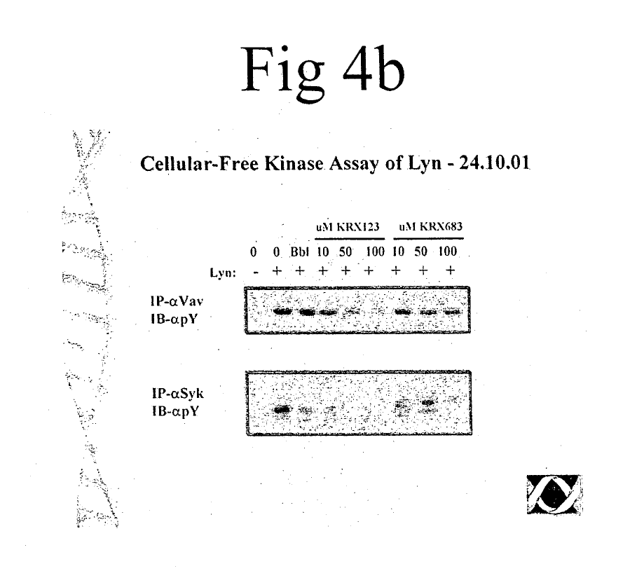

The results are shown in FIG. 4. These results show blots for three immunoprecipitates which are all substrates of Lyn: Lyn itself, CD19 and Syk and the level of phosphorylation is indicated in the absence of Lyn (0), and in the presence of Lyn (+) with increasing concentrations (0, 10, 50 and 100 μM) of the compound K055H302 in B-blac (see example 3). Phosphorylation level was determined with anti-phosphotyrosine.

As can be seen Lyn, CD19 and Syk all showed dose-dependent decreased phosphorylation in the presence of the compound of the invention, thus indicating that the compound is a true LAST inhibitor, as evident by a decrease in the level of its phosphorylation, and that its effects in vivo and in vitro, shown in the above examples were through inhibition of LAST.

### EXAMPLE 5

### Interruption of Interaction between Lyn and its Substrate Syk in the Presence of the Compound of the Invention.

For proving that the compound of the invention K055H302, comprising a Lyn derived peptide, blocks the complexation of Lyn with its substrate Syk the amounts of Syk, present in association with Lyn were measured in the presence of varying concentrations of the compound, by co-immuno-precipitation (co-IP).

WEHI-231 cells were incubated with 10, 50 and 100 mM of the compound K055H302 for 2 hours and following stimulation with a-IgM

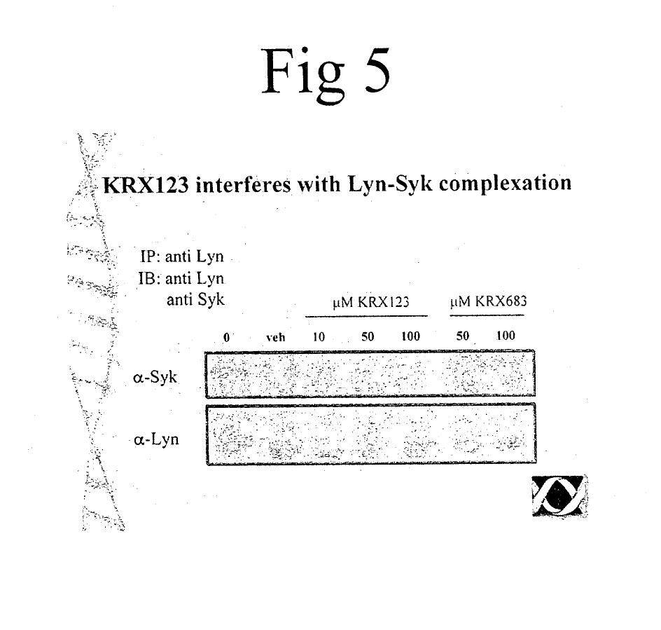

The Lyn was than immunoprecipitated using suitable anti-Lyn antibodies. The Lyn-immunoprecipitate was co-immunoreacted with anti-Syk antibodies. The results are shown in FIG. 5, which demonstrates that Syk levels in the Lyn-immunoprecipitates decreased in a dose dependent manner (the amounts of the Lyn itself were not changes).

These results support the theory of the invention that the compound comprising the Lyn-derived peptide interrupts the interaction of the Lyn and its substrate (Syk) as can be seen by the decrease in the amount of Syk complexed with the Lyn. An irrelevant compound comprising a peptide derived from the HJ-loop region of another kinase (GRK) termed “683” showed no effect.

### EXAMPLE 6

### Expression of the Lyn-Kinase, as Determined by Western Blots, in Various Transformed Cell Lines

In order to determine Lyn expression in various cancer cell lines, western blots of the cell lines were preformed using anti-Lyn antibodies.

Exponentially growing cells were collected and lysed in buffer containing 1% Nonidet P-40, 150 mM NaCl, 50 mM Tris-HCl (pH 8), 2 mM EDTA, 2 mM Na orthovanadate, 20 mM NaF, 5 mM Na pyrophosphate, 20 mM b-glycerophosphate, and ×1 protease inhibitor cocktail (Sigma P-8340). Lysates were cleared by centrifugation at 20,000 g for 15′ at 40° C., and protein concentrations were determined by the Bradford protein assay. Total cell extracts, normalized for protein concentration, were separated by SDS-PAGE, and immunoblotted (according to standard methods) with anti-Lyn antibodies. The anti-Lyn antibody used (SC-15) was purchased from Santa Cruz Biotechnology (Santa Cruz, Calif.).

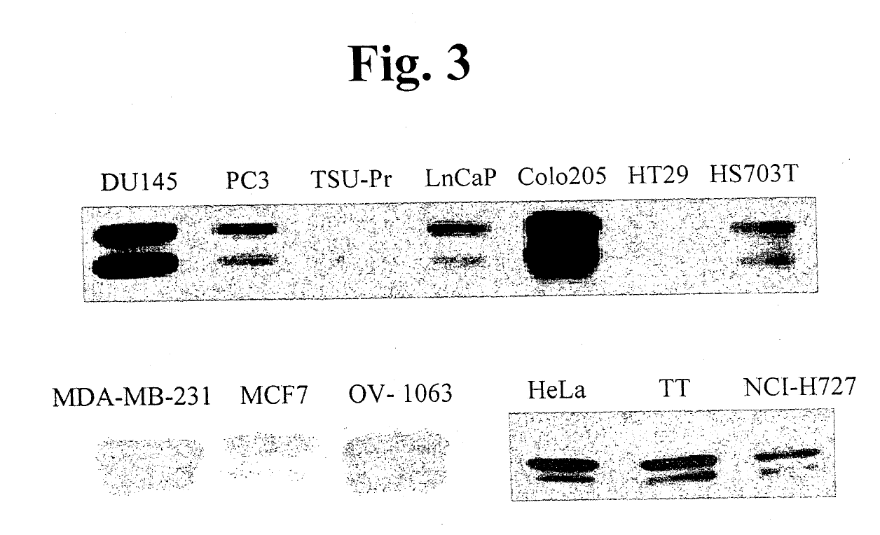

The results are shown in FIG. 3. As can be seen Lyn was expressed in the following cells lines: DU145 and PC3 (prostate cancer hormone independent, TSU-Pr (prostate cancer), LnCap (prostate cancer androgen dependent), Colo205 (colon cancer), HS703T (colon cancer) MDA-MB-231 (breast human cancer), MCF7(breast human cancer) OV-1063 (ovary cancer), Hela (cervix carcinoma, NC1—H727 (lung cancer), TT (carcinoma of the thyroid), indicating that all these cells express significant amounts of the Lyn, which associated signal transduction can be modulated by the compounds of the invention.

### EXAMPLE 7

### Immunohistochemistry and Tissue Micro-Arrays.

Formalin fixed paraffin tissues were immunohistochemically stained with anti Lyn polyclonal Ab (sc-15, Santa Cruz Biotechnology, Santa Cruz, Calif.), using the LAB-SA detection system (Zymed Laboratories, San Francisco, Calif.). Control staining for specificity was carried out in the presence of a Lyn-specific blocking peptide K055H302. Prostate cancer tissue arrays (PR200), and cancer screening array (CS200) were purchased from Clinomics Inc. (Pittsfield, Mass.). In all experiments a negative control was included.

Detailed Protocol:

1. Xylen deparaffinization 3×5 min.

2. 100% ethanol 3×2 min.

3. 96% ethanol 2×2 min.

4. Rinse with DDW 3×2 min

5. 15 min. in 3% H₂O₂ to inactivate endogenous peroxidase

6. Rinse with DDW 3×2 min

7. Antigen retrieval: microwave, 15 min at 92° C. (citrate buffer pH 6.0)

8. Cool to ˜50° C.

9. Rinse with DDW 3×2 min

10. Rinse with PBS 3×3 min.

11. Primary antibody (Lyn) [in 1:1 solution A (serum blocking solution): PBS]. Incubate 1 h at RT in humid environment.

12. Rinse with PBS 3×3 min.

13. Biotinylated 2nd antibody 20 min RT in humid environment

14. Rinse with PBS 3×3 min.

15. Enzyme conjugate (strepavidin-peroxidase-conjugate)-20 min. in a humid environment

16. Rinse with PBS 3×3 min

17. AEC (chromogen) staining-5-10 min.

18. Rinse with DDW 3×3 min

19.1 min. stain with Hematoxilin

20. Rinse with DDW 3×3 min.

21. Cover with mounting solution

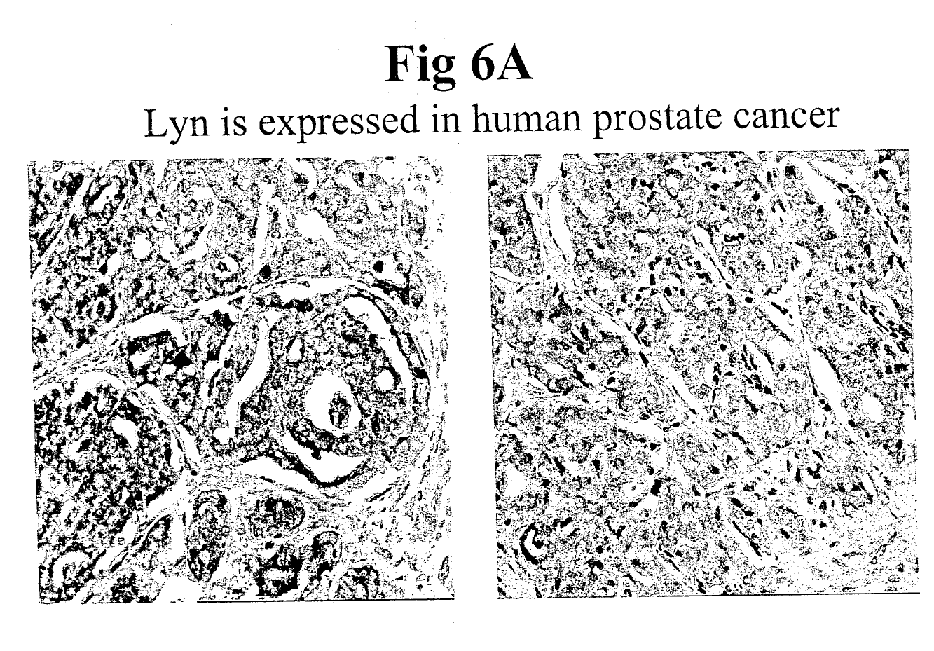

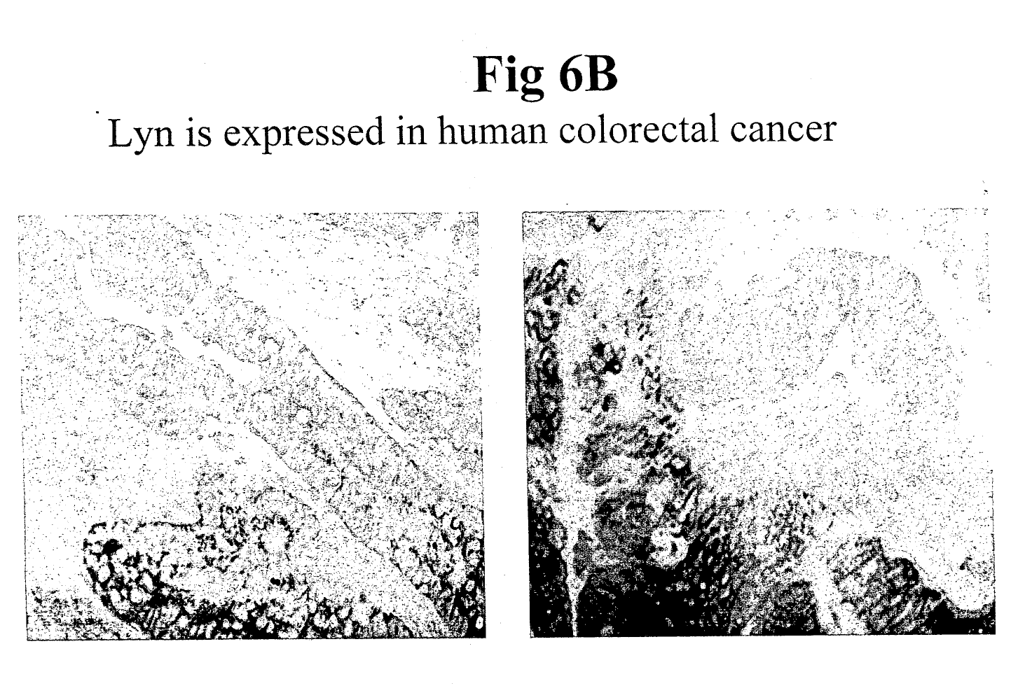

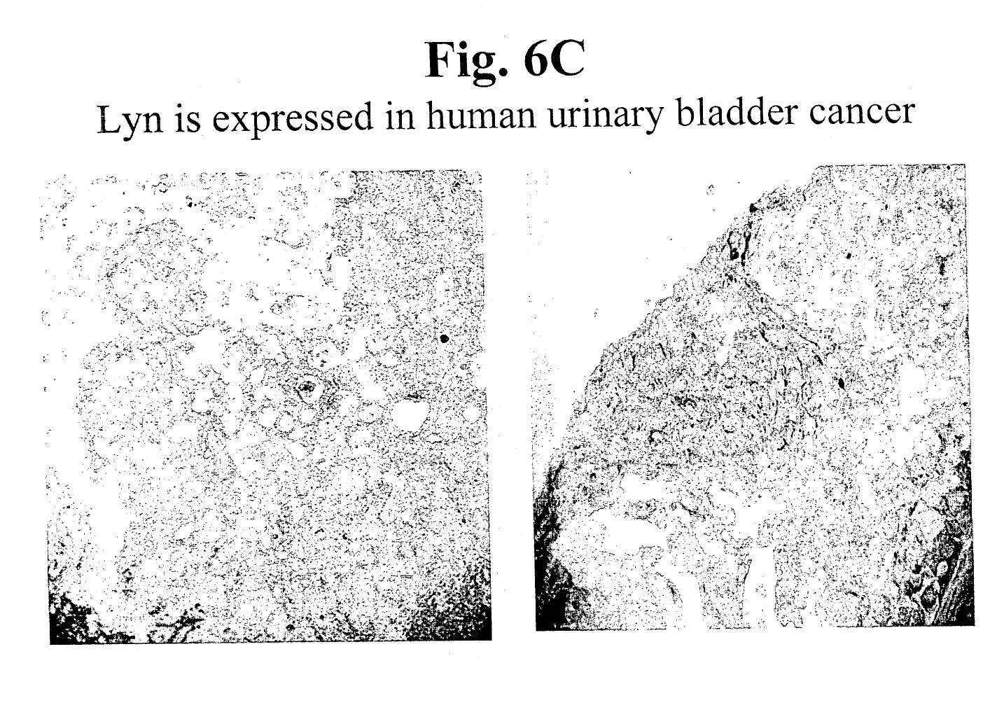

The results are shown in FIGS. 6A-6C wherein FIG. 6A: immunohistochemical staining of human prostate cancer specimens, showing two different samples of primary prostate cancer (original magnification ×200).

FIG. 6B shows: immunohistochemical staining of two human colorectal adenocarcinoma specimens (original magnification ×400).

FIG. 6c shows immunohistochemical staining of two human urinary bladder cancer specimens (transitional cell carcinoma, original magnification ×400).

As can be seen, in all these sections, Lyn antibodies showed detectable staining indicating that Lyn is present in all these cancer sections. In addition Lyn stained sections obtained from breast cancer, ovarian cancer and endometrium cancer patients (data not shown).

### EXAMPLE 8

### Inhibition of LAST by Small Interference RNA (siRNA)

For preparing Lyn-siRNA the following three Lyn-siRNA duplexes, derived from human Lyn, Acc. M16038 were obtained from a commercially available source:

1. Lyn423: positions 423-443 of Lyn (position relative to ATG: 125). Rhodamine labeled (sense strand).

2. Lyn436 positions 436-456 of Lyn (position relative to ATG:138). Rhodamine labeled (sense strand).

3. Lyn488: positions 488-508 of Lyn (positions relative to ATG:191)

For negative control, non-silencing siRNA (the sequence in the KLASER siRNA) was used. An additional negative control was Lamin siRNA.

Protocol and Reagents:

For 24-well, seed 3×104 cells 18 h before transfection, in RPMI without Antibiotics (with serum). The day after transfect with 0.9 ug siRNA as follows:

| | | | | | | Vol of |
|---|---|---|---|---|---|---|
| | | SiRNA | | | | OPTIMEM |
| | SiRNA | duplex | | | | added to |
| | duplex | Volume | OPTIMEM | OLIGOFECTAMINE | OPTIMEM | complexes |
| Format | (μg) | (μl) | (μλ) | (μl) | (μl) | (μl) |
| 24-well | 0.9 μg | 3 μl | 50 μl | 3 μl | 12 μl | 32 μl |
| | (60 pmole) | | | | | |

Protocol:

In tube 1, mix 3 μl SiRNA duplex with 50 μl OPTIMEM.

In tube 2, mix 3 μl OLIGOFECTAMINE with 12 μl OPTIMEM.

Incubate at RT for 7-10 min

Combine the 2 solutions, and gently mix by inversion.

Incubate at RT for 20-25 min. The solution may turn turbid.

Add 32 μl OPTIMEM to obtain final volume of 100□1

Add 100 μl to cells

Incubate fothe appropriate time needed

Results:

a) siRNA Lowers Lyn Expression in DU-145 Cells

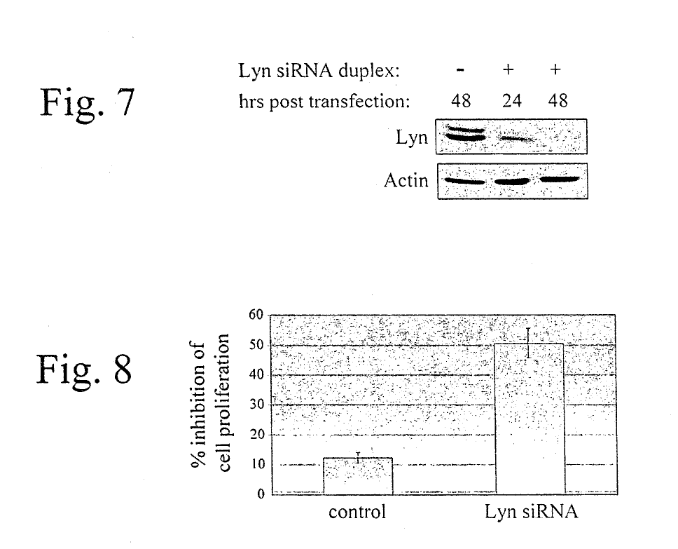

Prostate cancer cell line DU145 was transfected with Lyn423 siRNA according to the manufacture's protocol for 24 or 48 hours. The levels of actin and Lyn mRNA were than determined using a northern blot. The results are shown in FIG. 7. As can be seen Lyn siRNA caused a time dependent reduction in the amount of Lyn mRNA, while it had no effect on actin mRNA levels. Non-relevant siRNA had no effect on Lyn levels (data not shown).

b) siRNA Inhibits DU-145 Cell Proliferation

Prostate cancer cell line DU-145 was transfected with Lyn423 siRNA according to the manufacture's protocol. The number of cells was assessed by a cell counting assay 72 hours after incubation as described in example 4 above. The results are shown in FIG. 8 which represents four independent experiments each carried out in quadruplicates. Non-relevant siRNA served as control. As can be seen while non-relevant siRNA caused about 10% inhibition of cancer cell proliferation, the siRNA Lyn, caused about a 50% inhibition of proliferation.

c) Induction of Apoptosis by siRNA in DU-145 Cells:

Cultured DU-145 cells were seeded on cover slips and treated for 14 hours with siRNA of Lyn or of a non-relevant duplex. For determination of apoptosis the cells were stained PI and Tunnel staining. The staining results (data not shown) indicate that siRNA obtained from Lyn caused as significant apoptosis while non-relevant siRNA had no significant effect.

d) siRNA of Lyn Inhibits Phosphorylation of ERK in DU145 Cells.

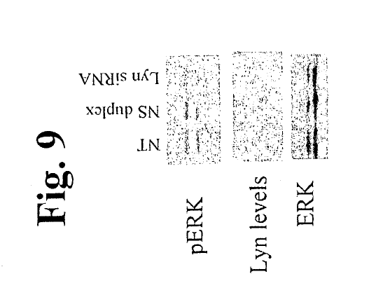

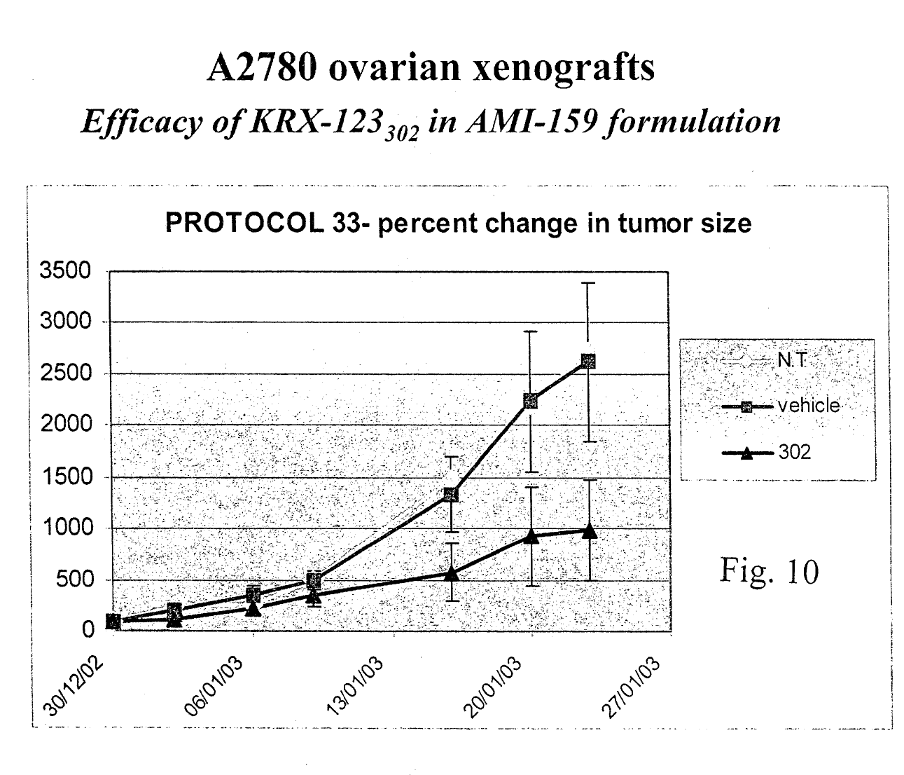

Prostate cancer cell line DU-145 was transfected with Lyn423 siRNA. according to the manufacture's protocol. The level of phosphorylation of ERK (pERK) as compared to not phosphorylated ERK was determined as described in example 4 with anti-phosphotyrosine antibodies, 72 hours after transfection. The results are shown in FIG. 9. As can be seen only siRNA of Lyn, and not treated or non-relevant siRNA (NS duplex) caused a marked decrease in the phosphorylation level of one of Lyn's downstream substrate ERK.

### EXAMPLE 9

### Inhibition of Ovarian Cancer by the Compound of the Invention

The ovarian cancer cell line, A270, was grown in RPMI-1640 culture medium with 10% fatal calf serum plus penicillin (100 U/ml), streptomycin (100 μg/ml), glutamine (2 mM) (see Example 2). The ovarian cells were harvested and injected subcutaneously into male mice strain CD1 of about 6-7 weeks of age, 5×10⁶ cells per mouse. After about 6 to 8 weeks, when the tumors became palpable, treatment of these mice was started by i.p. injection of 50 mg/kg of a solution comprising either compound K055H302 as detailed in FIG. 1. The compound solutions were prepared by taking pluronic based formulation AM1159 (see example 3). Tumor volume was measured twice a week. The results in FIG. 9 show a marked decrease in tumor size in the treated group as compared to untreated control and vehicle treated group. As can be seen the tumor diminishes in size with time when compound injections are administered. By contrast, the tumors in control animals grow exponentially over the same time period. Clearly, the compound had a very significant effect on the decrease of the size of ovarian cancer.

What is claimed is:

1. A method for the reduction of the growth of cancer cells the method comprising:
contacting the cells with an effective amount of a compound comprising a sequence selected from:
(a) a sequence which is a continuous stretch of at least five amino acids present in Lyn in positions 434-458 (HJ loop);
(b) a sequence which is a continuous stretch of at least five amino acids present in Lyn in positions 318-336 (αD region);
(c) a sequence which is a continuous stretch of at least five amino acids present in Lyn in positions 305-316 (B4-B5 region);
(d) a sequence which is a continuous stretch of at least five amino acids present in Lyn in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequences of (a) to (d) are maintained unaltered in the variant, and provided that the variant maintains the biological activity of the parent sequences of (a) to (d);
(f) a sequence of any one of (a) to (e) wherein at least one of the amino acids is replaced by the corresponding D-amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h).

2. A method for the treatment of cancer in an individual comprising administering to an individual, in need of such treatment, a therapeutically effective amount of a compound comprising a sequence selected from:
(a) a sequence which is a continuous stretch of at least five amino acids present in Lyn in positions 434-458 (HJ loop);
(b) a sequence which is a continuous stretch of at least five amino acids present in Lyn in positions 318-336 (αD region);
(c) a sequence which is a continuous stretch of at least five amino acids present in Lyn in positions 305-316 (B4-B5 region);
(d) a sequence which is a continuous stretch of at least five amino acids present in Lyn in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequences of (a) to (d) are maintained unaltered in the variant, and provided that the variant maintains the biological activity of the parent sequences of (a) to (d);
(f) a sequence of any one of (a) to (e) wherein at least one of the amino acids is replaced by the corresponding D-amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h).

3. A method according to claim 1 or 2, wherein the compound comprises a sequence of (a)-(HJ-loop), (e), (f), (g) and (h) as defined in claim 1.

4. A method according to claim 3, wherein the sequence of (a) is in positions selected from: 436 to 441, 441-453 and 447-456 of the Lyn.

5. A method according to claim 3, wherein the variant of (e) is produced by a combination of substitutions and chemical modifications.

6. The method of claim 1 or 2, wherein the compound is selected from the group of compounds specified in FIG. 1A or FIG. 1B.

7. A method according to claim 1 or 2, wherein the compound comprises a moiety for transfer across cell membranes in association with the sequence of any one of (a) to (i).

8. A method according to claim 7, wherein the moiety is a hydrophobic moiety.

9. A method according to claim 1 or 2 wherein the cancer is selected from:
carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, myeloid lymphoma, leukemia, acute myelocytic leukemia, chronic leukemia, Hodgkin's lymphoma, non-Hodgkin's lymphoma and metastasis of all the above.

10. A method according to claim 9 wherein the cancer is selected from: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, myeloid lymphoma, leukemia, acute myelocytic leukemia, chronic leukemia, Hodgkin's lymphoma′ non-Hodgkin's lymphoma and metastasis of all the above.

11. A method for the reduction of the growth of cancer cells from solid tumors comprising: contacting the cells with an inhibitor of Lyn-associated signal transduction (LAST), whereby said contact results in a reduction of growth of said cells.

12. A method of treatment of solid tumor in an individual, comprising:
administering to an individual, in need of such treatment, a therapeutically effective amount an inhibitor of Lyn-associated signal transduction (LAST), wherein said administration results in a reduction or stasis of said solid tumor.

13. The method of claim 11 or 12, wherein the LAST inhibitor is selected from the group consisting of:
(i) a compound comprising a sequence selected from:
(a) a sequence which is a continuous stretch of at least five amino acids present in Lyn in positions 434-458 (HJ loop);
(b) a sequence which is a continuous stretch of at least five amino acids present in Lyn in positions 318-336 (αD region);
(c) a sequence which is a continuous stretch of at least five amino acids present in Lyn in positions 305-316 (B4-B5 region);
(d) a sequence which is a continuous stretch of at least five amino acids present in Lyn in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);
(f) a sequence of any one of (a) to (e) wherein at least one of the amino acids is replaced by the corresponding D-amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(ii) a combination of two or more of the sequences of (a) to (h);
(iii) a compound comprising an antibody, or antigen-binding portion thereof, reactive with Lyn wherein said compound is capable of penetrating through cellular membranes; or an expression construct capable of expressing said antibody;
(iv) an antisense nucleic acid sequences complementary to a region in the Lyn gene or Lyn mRNA, so that hybridization between said antisense and said gene or hybridization between said antisense and said RNA, results in decrease in expression of Lyn;
(v) a small interfering RNA (siRNA) being complementary or identical to a region in the Lyn mRNA so that hybridization of said siRNA and the Lyn mRNA results in degradation of the Lyn mRNA;
(vi) a ribozyme that specifically cleaves Lyn RNA
(vii) an expression constructs coding for dominant negative Lyn; and
(viii) small organic molecules capable of inhibiting Lyn.

14. The method of claim 13, wherein said small organic molecule is a pyrazolo pyrimidine-type inhibitor.

15. The method of claim 13, wherein said compound of (i) is selected from the group consisting of the compounds depicted in FIG. 1A or FIG. 1B.

16. A method according to claim 11 or 12 for the treatment of a disease selected from carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, and metastasis of all the above.

17. A method according to claim 16 wherein the disease is selected from:
carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, and metastasis of all the above.

18. A compound selected from the group consisting of the compounds depicted in FIG. 1A or FIG. 1B.
